(19) United States
(12) Patent Application Publication
(10) Pub. No.: US 2024/0384236 A1
(43) Pub. Date: Nov. 21, 2024

(54) 3D RECONSTITUTED BONE MARROW NICHE AND USES THEREOF

(71) Applicants: The University of Hong Kong, Hong Kong (CN); Advanced Biomedical Instrumentation Centre Limited, Hong Kong (CN)

(72) Inventors: Pui Barbara Chan, Hong Kong (CN); Hoi Lam Cheung, Hong Kong (CN); Yu Hung Anskar Leung, Hong Kong (CN); Jessica Evangeline Tan Kabigting, Hong Kong (CN)

(21) Appl. No.: 18/667,906

(22) Filed: May 17, 2024

Related U.S. Application Data
(60) Provisional application No. 63/467,291, filed on May 17, 2023.

(51) Int. Cl. C12N 5/077; A61K 45/06

(52) U.S. Cl. CPC C12N 5/0669; A61K 45/06; C12N 2501/155; C12N 2501/90

(57) ABSTRACT

Provided herein is a platform that supports the survival and phenotype maintenance of leukemia cells by co-culturing the cells with 3DON. The 3D osteogenic niche (3DON) mimics that in bone marrow to support AML cell survival in cultures before treatment with sample chemotherapy drug. The 3DON is comprised of osteogenically differentiated mesenchymal stromal cells (MSCs) microencapsulated in 3D microspheres made from extracellular matrix (ECM). Also provided is a method that increases the chemo-sensitivity of leukemia cells by co-culturing them with 3DON. The platform may be used for drug screening applications and the 3DON may be used as an adjuvant for chemotherapy.

### CROSS-REFERENCE TO RELATED APPLICATION

This application claims the benefit of and priority to U.S. Provisional Application No. 63/467,291 filed May 17, 2023, the entire content of which is incorporated herein by reference for all purpose in its entirety.

### FIELD OF THE INVENTION

The disclosed invention is generally in the field of leukemia cells and 3D osteogenic niche (3DON) which recapitulate the leukemia bone marrow microenvironment. More specifically, it relates to the composition, materials, and methods of a novel 3DON that consists of stromal cells microencapsulated in extracellular matrix (ECM).

### BACKGROUND OF THE INVENTION

Acute myeloid leukemia (AML) is one of the most common acute leukemia (Brunetti, Gundry, and Goodell 2019). It is the most lethal hematopoietic cancer with a 5-year survival rate at around 27% (Duy et al. 2021; Marofi et al. 2021). AML has over 24 genetically defined subtypes (Vetrie, Helgason, and Copland 2020), making it a very heterogeneous disease and hence causing poor prognosis (HoushmandMohammad et al. 2017; Stanchina et al. 2020). Currently, the standard treatment for AML is a 7+3 regimen (7 days of cytarabine with 3 days of anthracycline) but it only results in 50% overall survival (Bertoli et al. 2017). This is followed by consolidation chemotherapy and hematopoietic stem cell transplantation for some high-risk patients (Yeung and Radich 2017). Relapse caused by chemoresistant leukemic cells occurs in more than 70% of patients (Villatoro et al. 2020). For aged individuals, who are the majority of AML patients, they may not tolerate the intensive chemotherapy or respond to the treatments due to their higher risk in complex karyotype and TP53 mutation (Stanchina et al. 2020). With the recent advancement in AML pharmacotherapies, tailor-made treatment for patients, especially the elderly, could be made possible by personalized medicine.

The bone marrow is a viscous tissue within the bone cavity responsible for hematopoiesis (Shafat et al. 2017). The two dynamic bone marrow niches-osteogenic niche (ON) that are made up of osteoblasts, osteocytes and osteoclasts; and perivascular niche, consist of endothelial cells and megakaryocytes, interact to regulate the functions of hematopoietic stem cells (HSCs) (Wang and Zhong 2018). Mesenchymal stromal cell (MSC) is an important cell type as they establish the hematopoietic environment by differentiating into many essential components of the BM niche, and they also affects the proliferation, differentiation, and homing of HSCs (Ladikou et al. 2020).

Leukemic stem cells (LSCs) are the precursors of AML cells which are genetically mutated from HSCs (Quek et al. 2016). In fact, leukemic cells interact with the BM microenvironment by activating bone morphogenetic protein 2 (BMP-2) and Smad1/5 signaling in MSCs to induce osteogenic differentiation, hence create a niche rich in pre-osteoblast to become a pro-tumoral environment (Ladikou et al. 2020) (Battula et al. 2017). However, one obstacle limiting AML for in vitro experimental study and drug screening application is that unlike AML cell lines, primary AML cells rapidly undergo spontaneous or stress-induced apoptosis in ex vivo cultures, which is highly patient dependent due to heterogeneity (Aasebø et al. 2021) (Brenner et al. 2019) (Cucchi et al. 2020). Opposingly, AML cells has prolonged survival in vivo (Abdul-Aziz et al. n.d.). This discrepancy between the in vitro and the in vivo behavior of AML cells, in particular, the spontaneous apoptosis and the poor survival of the AML cells in cultures (Ryningen et al. 2006) may be attributable to the lack of the pro-tumoral BM microenvironment consisting of soluble factors such as growth factors, soluble adhesion molecules and cell-cell/cell-extracellular matrix (ECM) interactions.

Different methods have been developed to enable ex vivo culture of primary AML cells (Cucchi et al. 2020), including the development of engineered conditioned medium with stem cell factor (SCF), granulocyte colony-stimulating factor (G-CSF), interleukin-3 (IL-3) (Lam et al. 2016) (Brenner et al. 2019); liquid tumor models such as 2D co-cultures with MSCs or endothelial cells to mimic the BM niche by secreting cytokines (Schuringa and Schepers 2009) (van Gosliga et al. 2007); and 3D co-cultures such as MSC-coated ceramic scaffolds (Antonelli et al. 2016), collagen I or fibronectin coated polylactic-co-glycolic acid (PLGA) and polyurethane (PU) scaffolds (Blanco et al. 2010), polyethylene glycol (PEG) hydrogels with HUVEC and MSCs (Bray et al. 2017). However, the preparation of the engineered conditioned medium is tedious, time-consuming, costly and inefficient because viral transfection of three different cell lines to over-express three different cytokines are necessary for producing the required medium (Hogge et al. 1996). Furthermore, the engineered conditioned medium and the 2D co-culture environment failed in mimicking the cell-cell or cell-ECM contacts, whereas the existing 3D cultures in a wide range of materials, although being more physiologically relevant, are yet to recapitulate the pro-tumoral osteogenic niche (Cucchi et al. 2020).

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### BRIEF SUMMARY OF THE INVENTION

Provided herein is a platform that comprised of leukemia cells co-cultured with biomimetic 3DON that support the survival of the leukemia cells. The 3DON is formed by encapsulating MSCs in naturally occurred ECM and differentiated towards osteogenic lineage. The 3DON is co-cultured with primary AML cells to support the survival, reduce the apoptosis and maintain the phenotype of AML cells for the applications of drug screening and chemotherapy adjuvant.

The composition of 3DON uses a formulation including cell component and ECM. In the preferred embodiment, the cell component that present in the bone marrow microenvironment, interact with the ECM and the co-culture cells, is healthy bone marrow (BM) derived MSCs or AML-derived MSCs, or other stromal cells that are present in the bone marrow microenvironment such as osteoblast, osteoclast, osteocyte, endothelial cells, adipocytes and immune cells. In the preferred embodiment, the natural ECM, being capable of providing support to the cells and interact with the cells, permitting cell migration and penetration, is collagen, or other material that supports cell growth and migration such as fibronectin, laminin and Matrigel. In the preferred embodiment, the cells being co-cultured with 3DON, is primary AML cells, or other leukemia cells including primary cells or cell lines.

The method of fabricating 3DON includes mixing of collagen, sodium hydroxide, culture medium and cells in a specific order, ratio, pH, concentration and volume; the temperature and incubation time is also controlled for gelation. The 3DON is followed by osteogenic differentiation which includes the use of osteogenic induction medium prepared with dexamethasone, β-glycerophosphate, ascorbic acid and bone morphogenic protein 2 (BMP2) in a specific concentration. In addition, the culture plate for forming 3DON is coated with pluronic F127 to reduce cell attachment. Leukemia cells were seeded into a well containing 3DON for a period of time before screening.

The novel 3DON, when being co-cultured with primary AML cells, can increase the percentage of viable AML cells (60-70%) and reduce the percentage of AML apoptotic cells (5-10%). The phenotype of primary cells (CD33+ and CD34−) can also be preserved, which can be explained by the cell-cell and cell-ECM interaction between AML blasts and the 3DON, and secretion of cytokines by 3DON. Apart from this, drug test demonstrated that healthy 3DON can increase the chemosensitivity of primary AML cells towards daunorubicin and doxorubicin. AML cells were less sensitive to daunorubicin and doxorubicin upon co-culture with AML MSC-derived niches.

In summary, the use of novel 3DON for primary AML cell co-culture tackled the problem of spontaneous apoptosis of primary AML cells in ex vivo cultures and allowed the development of personalized drug screening that provide valid results. On the other hand, the 3DON increases the chemosensitivity of primary AML cells such that it can be applied as an adjuvant to chemotherapy by replenishing healthy BM-MSC derived 3DON into the bone marrow of the AML patients.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIGS. 1A-1C show a schematic diagram of the overall experimental and drug screening design. FIG. 1A shows preparation of the nine groups (three control groups: negative control 1 (NC1), negative control 2 (NC2), positive control (PC) and six experimental groups: 2D mesenchymal niche (2DMN), 3D mesenchymal niche (3DMN) and 3D osteogenic niche (3DON) for AML co-culture using BM-MSCs derived from healthy donors and AML patients; FIG. 1B shows subsequent evaluation on viability, apoptosis, cytokine secretion, phenotype maintenance and drug response; FIG. 1C shows workflow for high-throughput personalized drug screening. Cells were isolated from the blood samples. The AML cells were co-cultured with the collagen encapsulated patient-derived MSCs (3DMN) or co-cultured with 3DMN that has undergone osteogenic differentiation (3DON) for drug testing with the assistance from robotic dispenser. Drug test results were generated and analysed.

FIGS. 2A-2G show the characterization of the 3DON and 3DMN. FIGS. 2A-2B are images that show ALP and von Kossa staining of (FIG. 2A) healthy 3DON and healthy 3DMN; and (FIG. 2B) AML 3DON and AML 3DMN cross-section with magnified view on the bottom (I, II: 3DON; III, IV: 3DMN); FIG. 2C show scanning electron microscopy images of (I-III) 3DON and (IV-VI) 3DMN to reveal the microstructure. Magnification: (I, IV: 4kX; II, V: 20kX, and III, VI: 100 kX) circles indicate cells, asterisks indicate calcium deposits in the shape of granules; arrows indicate collagen fibers; FIG. 2D demonstrate EDX analysis of 3DON. Elemental analysis (IV, IX) refers to the region highlighted in (I, VI), EDX elemental mapping of the distribution of Calcium (II, VII) and Phosphorus (III, VIII); (V) element weight percentage (wt %) of calcium in 3DON and 3DMN and (X) phosphorus in 3DON and 3DMN. Unpaired t-test (calcium wt % in 3DON vs 3DMN and phosphorus wt % in 3DON vs 3DMN p<0.0001) (****p<0.0001). Error bars were ±SE; FIG. 2E show immunofluorescence staining of osteogenic markers osteopontin on the healthy 3DON (I) and AML 3DON (II) (scale bars: 200 μm). 3DON samples were collected on day 21 of osteogenic differentiation and 3DMN samples were collected 3 days after encapsulation in collagen. FIG. 2F are bright field images of the gross appearance of (I) healthy 3DON and (II) AML 3DON for drug screening tests. (III) Diameters of the 3DONs for drug screening tests. Mann-Whitney test (healthy 3DON vs AML 3DON p=0.007) (**p<0.01). Error bars were ±SE; FIG. 2G are live-dead staining on the healthy and AML 3DMN and 3DON (scale bars: 200 μm), with insets showing their 3D view.

FIGS. 3A-3C show the morphology of AML cells under various culture conditions. Images taken on day 2 of culture with magnified view on the bottom and insets showing individual cells (FIG. 3A: I: negative control 1 (NC1); II: negative control 2 (NC2); FIG. 3B: III: positive control (PC); IV: healthy 2D mesenchymal niche (2DMN); FIG. 3C: V: healthy 3D mesenchymal niche (3DMN); VI: healthy 3D osteogenic niche (3DON); VII: AML 2DMN; VIII: AML 3DMN; IX: AML 3DON) (black arrows: bright and rounded AML cells; white triangle: fragmented AML cells; black triangle: membrane ruffles on AML cells) (Scale bars: 500 μm for the original image and 100 μm for magnified view).

FIGS. 4A-4D show the viability and apoptosis of AML cells in different groups. The experimental groups were negative control 1 (NC1), negative control 2 (NC2), positive control (PC), healthy 2D mesenchymal niche (2DMN), healthy 3D mesenchymal niche (3DMN), healthy 3D osteogenic niche (3DON), AML 2DMN, AML 3DMN, and AML 3DON. FIG. 4A is a bar chart showing the cell number of AML cells via trypan blue exclusion test. One-way ANOVA test showed no significant (n.s.) difference in all groups p=0.804. Error bars were ±SE. FIG. 4B is a bar chart showing the viability of AML cells measured by trypan blue exclusion test. Kruskal Wallis p<0.0001, Dunn's post-hoc test (AML 3DMN vs NC2 p=0.0383, healthy 3DON vs NC2 p=0.0384, PC vs NC2 p=0.0480, AML 3DMN vs healthy 3DMN p=0.0437) (*p<0.05). Error bars were ±SE. FIG. 4C shows proportion of apoptotic cells analyzed by TUNEL assay. One-way ANOVA p=0.0017, Bonferroni's post-hoc test (NC2 vs healthy 3DMN p=0.006, healthy 3DMN vs AML 3DMN p=0.0014, healthy 3DMN vs AML 3DON p=0.0038, NC1 vs healthy 3DMN p=0.0373) (**p<0.01, *p<0.05)). Error bars were ±SE. FIG. 4D are representative flow cytometry plots of TUNEL assay, propidium iodide versus dUTP-FITC. Cells that were dUTP-FITC positive were considered apoptotic cells. The results were collected from 3 different biological experiments with technical triplicates.

FIGS. 5A-5C present the cytokine microarray assay. The presence of cytokines in the conditioned medium of different groups detected by cytokine arrays. FIG. 5A is a schematic representation of the cytokine spot positions. FIG. 5B are representative cytokine arrays (I: healthy 3DON; II: healthy 3DMN, III: healthy 2DMN, IV positive control (engineered growth factor cocktail medium for hematopoietic cells) collected from engineered murine cell lines; V: AML 3DON, VI: AML 3DMN, VII: AML 2DMN VIII: uncultured MSC medium as a negative control). FIG. 5C is a heatmap showing the fold change of cytokines in different groups. The cytokines were categorized according to their functions. The number in brackets showed the number of cytokines in that category. The results were collected from triplicated samples from 3 different biological experiments.

FIGS. 6A-6G show the analysis of the presence of cytokines in different culture groups. FIGS. 6A-6C are bar charts showing the normalized fold changes of the secreted cytokines in different culture groups. Kruskal Wallis test (p=0.0036) with Dunn's post hoc test, healthy 2DMN vs healthy 3DON, IL-8 (p=0.0219), GRO-α (p=0.0219), IL-6 (p=0.0219) and GRO (p=0.0219), healthy 3DMN vs healthy 3DON, OPG (p=0.0219) and TIMP-2 (p=0.0219). (*p<0.05). Error bars were ±SE. The experiments were conducted 3 times with different biological samples, each experiment was performed in triplicates. FIGS. 6D-6F are PANTHER GO-Slim analysis of the (FIG. 6D) protein class, (FIG. 6E) biological process, (FIG. 6F) pathways that the enriched cytokines in PC, healthy 3DON, AML 3DMN, and AML 3DON groups that the AML cells had the highest viability. FIG. 6G shows GO term enrichment analysis of the enriched cytokines in PC, healthy 3DON, AML 3DMN, and AML 3DON groups that the AML cells had the highest viability. The graph was produced by the online Gonet tool [71].

FIGS. 7A-7D show the flow cytometry analysis of the phenotypes of AML cells. FIG. 7A shows representative flow cytometry plots of CD34-FITC versus CD33-APC/Cy7. The experimental groups were day 0 control, negative control 1 (NC1), negative control 2 (NC2), positive control (PC), healthy 2D mesenchymal niche (2DMN), healthy 3D mesenchymal niche (3DMN), healthy 3D osteogenic niche (3DON), AML 2DMN, AML 3DMN, and AML 3DON. FIG. 7B is a bar chart showing CD33+ AML cell population analyzed by flow cytometry after being cultured under different conditions. FIG. 7C is a bar chart showing CD34+ AML cell population analyzed by flow cytometry after being cultured under different conditions. Kruskal Wallis test (p<0.0001) with Dunn's post-hoc test (D0 control vs NC1 p=0.0075, DO control vs NC2 p=0.0109, D0 control vs PC p=0.0002, D0 control vs healthy 2DMN p=0.0001, D0 control vs healthy 3DMN p<0.0001, D0 control vs healthy 3DON p=0.0076) (*p<0.05, **p<0.005, ****p<0.001, ****p<0.0001) Error bars were ±SE. FIG. 7D is a bar chart showing CD33+ CD34-AML cell population analyzed by flow cytometry after being cultured under different conditions. Kruskal Wallis test (p=0.0112) with Dunn's post-hoc test (D0 control vs PC p=0.0175, D0 control vs healthy 2DMN p=0.0112, D0 control vs healthy 3DMn p=0.0008) (*p<0.05, **p<0.005, ***p<0.001) Error bars were ±SE.

FIGS. 8A-8B present the drug test (daunorubicin) results in different culture conditions. FIG. 8A shows dose-response curves showing the percentage viability of primary AML cells under daunorubicin drug exposure in different culture conditions. Percentage viabilities of AML cells were plotted against concentration of daunorubicin. All values were averages of replicates normalized to 100%. Error bars were ±SD. FIG. 8B is a bar chart showing the IC₅₀ values for daunorubicin drug test results with primary AML cells under different culture conditions. Kruskal Wallis test (p<0.0001), Dunn's post-hoc test (NC1 vs PC p=0.0111, NC1 vs AML 2DMN p<0.0001, NC1 vs AML 3DMN p<0.0001, NC1 vs AML 3DON p=0.0002, NC2 vs PC p=0.0046, NC2 vs PC p=0.0046, NC2 vs AML 2DMN p<0.0001, NC2 vs AML 3DMN p<0.0001, NC2 vs AML 3DON p<0.0001, healthy 2DMN vs AML 2DMN p=0.0011, healthy 2DMN vs AML 3DMN p=0.0041, healthy 2DMN vs AML 3DON p=0.0107, healthy 3DMN vs AML 2DMN p=0.0485, healthy 3DON vs AML 2DMN p=0.0124, healthy 3DON vs AML 3DON p=0.0375). (*p<0.05, **p<0.005, ***p<0.001) Error bars were ±SE. The experiments were repeated three times independently, with each experiment performed in triplicate.

FIGS. 9A-9B present the drug test (doxorubicin) results in different culture conditions. FIG. 9A are dose-response curves showing the percentage viability of primary AML cells under doxorubicin drug exposure in different culture conditions. Percentages viability of AML cells were plotted against concentration of doxorubicin. All values were averages of replicates normalized to 100%. Error bars were ±SD. FIG. 9B is a bar chart showing the IC₅₀ values for doxorubicin drug test results with primary AML cells under different culture conditions. Kruskal Wallis test (p<0.0001), Dunn's post-hoc test (NC1 vs AML 2DMN p=0.0006, NC1 vs AML 3DMN p=0.0003, NC1 vs AML 3DON p=0.0005, NC2 vs AML 2DMN p=0.0006, NC2 vs AML 3DMN p=0.0003, NC2 vs AML 3DON p=0.0006) (*p<0.05, **p<0.005, ***p<0.001) Error bars were ±SE. The experiments were repeated three times independently, with each experiment performed in triplicate.

### DETAILED DESCRIPTION OF THE INVENTION

The disclosed method and compositions can be understood more readily by reference to the following detailed description of particular embodiments and the Example included therein and to the Figures and their previous and following description.

Disclosed are compositions, platforms, systems, and methods involving biomimetic 3D osteogenic niches (3DONs). In some forms, the 3DON is formed by encapsulating mesenchymal stromal cells (MSCs) in extracellular matrix (ECM) and differentiating the MSCs towards the osteogenic lineage. In some forms, the MSCs are bone marrow derived MSCs. In some forms, the MSCs are osteoblast cells, osteoclast cells, osteocytes, endothelial cells, adipocytes, immune cells, or combinations thereof. In some forms, the ECM is collagen, fibronectin, laminin, Matrigel, or combinations thereof.

In some forms, the 3DON are formed by mixing the ECM, the MSCs, sodium hydroxide, and culture medium and by inducing osteogenic differentiation. In some forms, ECM, MSCs, sodium hydroxide, and culture medium are mixed in a specific order, ratio, pH, concentration, volume, or combinations thereof. In some forms, the mixing is performed in a culture plate coated with pluronic F127. In some forms, the temperature and incubation time are chosen to allow for gelation. In some forms, the osteogenic differentiation is induced by use of osteogenic induction medium prepared with dexamethasone, β-glycerophosphate, ascorbic acid, and bone morphogenic protein 2 (BMP2) in a specific concentration.

Also disclosed are platforms comprising leukemia cells co-cultured with the disclosed 3DON. In some forms, the platform supports the survival of the leukemia cells. In some forms, the leukemia cells are primary acute myeloid leukemia (AML). In some forms, co-culturing the 3DON and leukemia cells supports the survival, reduces the apoptosis, and/or maintains the phenotype of the leukemia cells. In some forms, co-culturing the 3DON and leukemia cells increases the chemo-sensitivity of leukemia cells.

In some forms, the percentage of viable leukemia cells is increased and/or and the percentage of apoptotic leukemia cells is reduced in leukemia cells maintained in the platform as compared to leukemia cells not maintained in the platform. In some forms, the phenotype of primary cells (CD33+ and CD34−) is preserved to a greater extent in the cells maintained in the platform as compared to cells not maintained in the platform.

Also disclosed are methods of drug screening comprising exposing the leukemia cells of the disclosed platform to a test compound and comparing the effect of the test compound on the leukemia cells to a control platform that was not exposed to the drug.

Also disclosed are methods of treating a patient in need of chemotherapy with a chemotherapeutic and with the disclosed 3DON. In some forms, the 3DON is in a pharmaceutical formulation.

### A. Definitions

As used herein, “cells” refers to the smallest basic unit of an organism, which is typically microscopic and consists of cytoplasm enclosed in a membrane. “Cells” can be from cell lines or isolated from human or animals such as mouse, including but not limited to leukemia cells, MSCs, pre-osteoblasts, osteoblasts, osteoclasts, osteocytes, chondrocytes, endothelial cells, adipocytes and immune cells.

As used herein, “ECM” refers to the extracellular matrix materials, in pure, isolated, partially isolated, recombinant or synthetic form, including but not limited to collagen type I, II, IV, fibronectin, laminin, Matrigel, or a combination thereof.

### B. Materials for Fabrication of 3DON, Co-Culture and Drug Test

### 1. ECM Materials

The ECM used for fabricating 3DON must be able to provide support to the cells and interact with the cells to allow cell growth, permitting cell migration and penetration without introducing toxicity. The ECM used can be naturally occurring or synthetic such as collagen type I, II, IV, fibronectin, laminin, Matrigel, or a combination thereof. The ECM can be derived from either natural or synthetic sources, and it can be induced to solid form under specific conditions and support cellular survival and growth. The ECM can be produced from isolation or extraction from various animal sources, such as rat tail, porcine skin, bovine tendon, or human placenta.

### 2. Cell Components

The cells being microencapsulated in the 3DON can interact with the ECM surrounding them and the co-culture cells, they can be pre-osteoblasts, osteoblasts, osteoclasts, osteocytes, chondrocytes, endothelial cells, adipocytes and immune cells, preferably MSCs. The cells can survive and proliferate inside the 3DON. The cells can be derived from human bone marrow, peripheral blood, adipose tissue, umbilical cord tissue, amniotic fluid, or any other tissue or cells that can give rise to MSCs from healthy individuals or leukemia patients. The leukemia cells being co-cultured with 3DON can be of primary culture, or cell line culture, such as acute lymphocytic leukemia cells (ALL), chronic lymphocytic leukemia cells (CLL), chronic myeloid leukemia cells (CML), HL-60, KG-1, MOLM13 etc., preferably primary AML cells. The co-cultured cells can be obtained from peripheral blood or bone marrow from leukemia patients.

### C. Method of Fabrication of 3DON, Co-Culture and Drug Test

The 3DON is produced by encapsulating MSCs in an ECM, preferably rat-tail type I collagen. The MSC cell density can be 10000 cells/ml-300000 cells/ml preferably 50000 cells/ml. The collagen concentration can be 0.1 mg/ml-4 mg/ml preferably 2 mg/ml. The volume of 3DON can be 1 μl-200 μl, preferably 10 μl for drug test. The culture medium, IN sodium hydroxide, stock collagen and cell suspension are mixed according to this order and pipetted into the wells of a 384-well culture plate, or other culture plate such as 96-well or 1536-well plate without coating or pre-coated with pluronic F127 for an hour at room temperature. The 3DON is incubated at 37° C., 5% CO₂ for 15 minutes to 120 minutes, preferably 60 minutes for gelation. The solidified 3DON is cultured for 3 days in Dulbecco's Modified Eagle Medium-low glucose (DMEM-LG) supplemented with 10% fetal bovine serum (FBS), 1% antibiotic-antimycotic (anti-anti). Osteogenic differentiation of the encapsulated MSCs is performed immediately or after 1 to 7 days culture, preferably 3 days culture. The 3DON is cultured in DMEM-LG supplemented with 10% FBS, 1% anti-anti, 100 nM dexamethasone (Sigma), 50 μM ascorbic acid 2-phosphate (BioChemika), 10 mM b-glycerophosphate (Calbiochem) and 10 ng/ml BMP-2 (PeproTech) as the osteogenic differentiation induction medium. The 3DON is cultured for 7 to 28 days, preferably 21 days in a 37° C. 5% CO₂ incubator with regular medium replenishment every 2-3 days before co-culture.

The leukemia cells are then seeded to the wells containing 3DON with percentage of leukemia cells being 10%-90%, preferably 50% of the total cell number in the well. The medium used for culture is 1:1 mixture of DMEM-LG medium (supplemented with 10% FBS and 1% anti-anti) and Roswell Park Memorial Institute (RPMI) 1640 medium (supplemented with 20% FBS and 1% anti-anti).

Leukemia cells are co-cultured with the 3DON for 1 to 7 days, preferably 3 days. The drugs applied can be FDA approved for leukemia, such as chemotherapeutic drugs, or chemicals under development for treating leukemia patients. Drugs of 5-30 different concentrations are administered into the culture wells. The leukemia cells are exposed to drugs on any day of the co-culture, preferably the second day, for 0.5 hours-96 hours, preferably 24 hours in a 37° C. 5% CO₂ incubator and cells are aspirated into another culture plate for viability assay such as PrestoBlue, Resazurin assay, alamarBlue, (3-(4,5-Dimethylthiazol-2-yl)-2,5-diphenyltetrazolium bromide) (MTT) assay etc. Graphs plotting the response of AML cells versus the concentration of drug can be used for calculating IC₅₀ values for each type of drug.

### D. Effects of 3DON in AML Co-Culture

The 3DON is designed to mimic the bone marrow microenvironment, specifically the osteogenic niche, to support the survival and phenotype maintenance of primary AML cells. To test for the effects of 3DON in AML co-culture, nine culture groups are designed for comparison. Primary AML cells are cultured in three control groups, I: negative control 1 (AML medium) (NC1); II: negative control 2 (MSC medium) (NC2); and III: positive control (engineered growth factor cocktail medium for hematopoietic cells) (PC), and co-cultured in six experimental groups: IV: healthy 2D mesenchymal niche (MSC feeder layer) (2DMN); V: healthy 3D mesenchymal niche (3DMN); VI: healthy 3D osteogenic niche (3DON); VII: AML 2DMN; VIII: AML 3DMN; IX: AML 3DON. The NC groups are used to confirm that the differences between the co-culture groups and the NC groups are indeed due to the effect of the co-culture but not the effect of the medium. The engineered growth factor cocktail medium has been reported to significantly reduce apoptosis of AML cells. (Hogge et al. 1996; Lam et al. 2016) as it contains conditioned medium from engineered murine stromal cell lines that produce stem cell factor (SCF), granulocyte colony-stimulating factor (G-CSF), interleukin-3 (IL-3) and fms-like tyrosine kinase 3 ligand (FLT3 ligand). (Hogge et al. 1996; Lam et al. 2016) AML medium and MSC medium are the negative controls because of the absence of stromal cells and supplementation of cytokines. Human MSCs are used as the niche cells to support AML cultures in the six co-culture experimental groups. 2DMN is prepared by seeding MSCs as a 2D feeder layer. 3DMN is prepared by encapsulating MSCs in collagen microspheres. 3DON is prepared by differentiating the 3DMN into osteogenic lineage. The primary AML cells are cultured under different conditions and collected for subsequent evaluation of their viability, apoptosis, phenotype maintenance, and drug response. Medium from 3DON and PC before co-culture are also collected for cytokine secretion assay.

Results demonstrated that the reconstituted AML 3D BM niches derived from AML patients provided the optimal niche to support the primary AML in cultures. Specifically, the viability of AML cells co-cultured with the AML 3DMN and AML 3DON groups were the highest, while the percentages of apoptotic cells in them were the lowest. Cytokine array analysis revealed that the enhanced secretion of anti-apoptotic cytokines such as IL-8, GROα and OPG in the AML 3DMN and AML 3DON groups. In addition, the AML phenotype of CD33+ and CD34-were nicely preserved in these groups. The intrinsically high chemoresistance of AML was well retained by the AML niches (2DMN, 3DMN, and 3DON groups) as compared to the healthy niches and other control groups, using daunorubicin and doxorubicin as examples, suggesting the physiological resemblance of the AML niches and the necessity to reconstitute AML BM niches for valid drug screening. Moreover, the healthy BM niches (2DMN, 3DMN, and 3DON), while equally supporting the AML cell survival and phenotype maintenance in vitro, showed increased drug sensitivity to the two sample drugs used, indicating that reconstituting a healthy BM niche in leukemic patients may overcome the intrinsic chemoresistance problem in leukemia patients. This work contributes to the application of the AML 3DON in personalized drug screening and suggests the potential of using the healthy 3DON as a chemotherapeutic adjuvant for better AML chemotherapies with enhanced drug sensitivity.

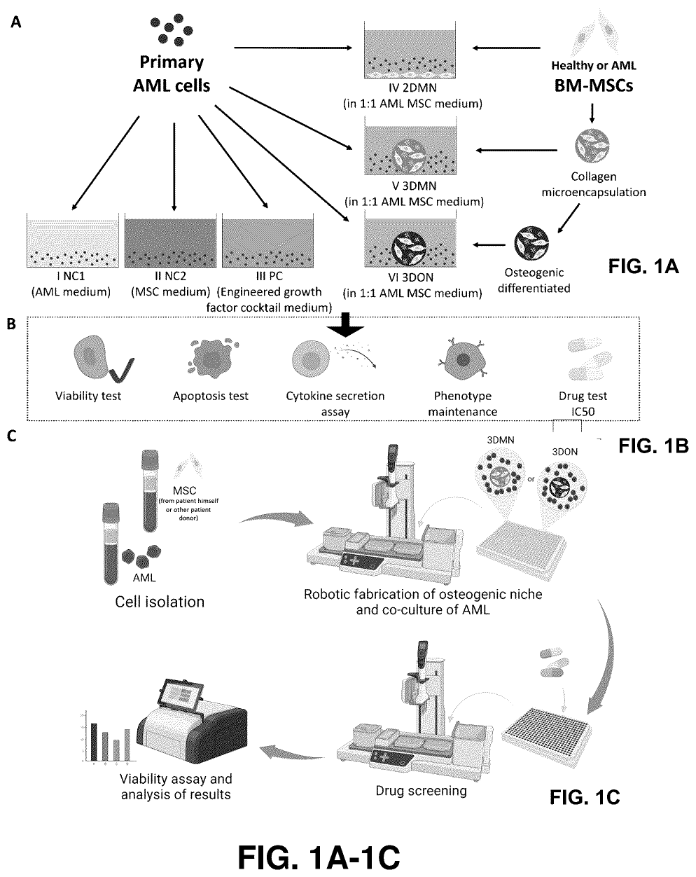

FIG. 1 shows a schematic diagram of the overall experimental and drug screening design. (A) Preparation of the nine groups (three control groups: negative control 1 (NC1), negative control 2 (NC2), positive control (PC) and six experimental groups: 2D mesenchymal niche (2DMN), 3D mesenchymal niche (3DMN) and 3D osteogenic niche (3DON) for AML co-culture using BM-MSCs derived from healthy donors and AML patients; (B) Subsequent evaluation on viability, apoptosis, cytokine secretion, phenotype maintenance and drug response; (C) Workflow for high-throughput personalized drug screening. Cells were isolated from the blood samples. The AML cells were co-cultured with the collagen encapsulated patient-derived MSCs (3DMN) or co-cultured with 3DMN that has undergone osteogenic differentiation (3DON) for drug testing with the assistance from robotic dispenser. Drug test results were generated and analysed.

### EXAMPLES

### Methods

### AML Cell Culture

AML primary cells were cultured as previously reported [20]. AML primary cells isolated from 6 patients (1 male and 5 females, ages 51-71) with FAB stage M1 or M5 were used, Department of Medicine, the University of Hong Kong. All procedures were approved by the Institutional Review Board of the University of Hong Kong/Hospital Authority Hong Kong West Cluster (HKU) (UW 05-183 T/846). Informed written consent from all participants was obtained prior to conducting the research. Primary cells were maintained in primary culture medium which was composed of 50% Iscove's Modified Dulbecco's Medium (IMDM) full medium and 50% conditioned medium collected from engineered murine fibroblast cell lines [20]. IMDM full medium was prepared from IMDM supplemented with 10% fetal bovine serum (FBS) (Gibco) and 1% antibiotic-antimycotic (Gibco). The primary cells were thawed in primary culture medium and immediately seeded in different culture conditions.

### MSC Cell Culture

To discover the differences in behaviours of AML cells in healthy niche environment and AML niche environment, human bone marrow-derived mesenchymal stem cell or stromal cells (MSCs) from 3 healthy donors (M, 38 years old; M, 25 years old; F, 24 years old) and 1 AML patients (F, 54 years old) were obtained. All procedures were approved by the Institutional Review Board of the University of Hong Kong/Hospital Authority Hong Kong West Cluster (HKU) (UW 05-183 T/846) The cells were grown in Dulbecco's modified Eagle medium-low glucose (DMEM-LG) (Gibco), supplemented with 10% FBS, 1% antibiotic-antimycotic and 1% GlutaMax (Gibco). MSCs were maintained in humidified conditions with 5% CO₂ at 37° C. and medium was refreshed every 3-4 days until 80-90% confluence. Cells were trypsinized using 0.05% trypsin-EDTA (Gibco) and cells at passage 6 were used for microencapsulation.

### Formation of 3DMN

To mimic the 3D bone marrow niche consisting of mesenchymal cells, 3DMN were prepared as reported previously [29]. Briefly, rat-tail collagen type I (BD Biosciences) was neutralized with IN sodium hydroxide (Sigma-Aldrich) and MSCs were suspended in the neutralized collagen solution in an ice bath. The cell mixture was diluted with culture medium to reach a cell density of 5×10⁵ cells/ml and a collagen concentration of 2 mg/ml. Cell mixture containing 5×10⁴ cells was dispensed into 96 well plates pre-coated with Pluronic F-127 (Sigma) to make the plate non-adhesive such that the MSCs would not migrate from the 3DMN to attach at the bottom of the plate (for flow cytometry and viability test), and 5×10³ cells in 384 well plates also pre-coated with F-127 (for drug test). They were then incubated at 37° C. 5% CO₂ environment for 45 minutes to induce gelation. The collagen microencapsulated MSC produced is the 3DMN. Healthy 3DMN was formed by healthy MSCs while AML 3DMN was composed of AML-patient derived MSC. The 3DMN was ready for subsequent experiments after 3 days of culture.

### Formation of 3DON

To mimic the 3D bone marrow niche that consists of osteolineage cells, 3DON was prepared as follows. The 3DON was formed from the osteogenic differentiation of the 3DMN 3 days after cell encapsulation as previously described [31]. The 3DMN was cultured in DMEM-LG supplemented with 10% FBS, 1% antibiotic-antimycotic, 100 nm dexamethasone (Sigma), 50 μm ascorbic acid 2-phosphate (BioChemika), 10 mm b-glycerophosphate (Calbiochem) and 10 ng/ml BMP-2 (PeproTech) as the osteogenic differentiation induction medium. The 3DMN was cultured for 21 days with osteogenic medium replenishment every 2-3 days before co-culture. These 3DMN that had undergone osteogenic differentiation were called 3DON. Healthy 3DON was formed by healthy MSCs while AML 3DON was composed of AML-patient derived MSC. The 3DON was ready for subsequent experiments once completed 21 days of osteogenic differentiation.

### Preparation of 2DMN Layer

To compare the difference between 2D and 3D culture, 2DMN was prepared by seeding 5×10³ cells into each well of a 384-well plate, and 5×10⁴ cells into each well of a 96-well plate. The seeded cells were cultured in DMEM-LG for 3 days before co-culture with AML cells.

### Co-Culture & Cell Viability Analysis

To observe how different niches affect the viability, apoptosis, and phenotypes of AML cells, co-culture experiments were performed. For the co-culture experiment, there were six co-culture groups, healthy and AML 3DON, 3DMN and 2DMN, the control groups included AML cultured in AML cell line medium (medium (Roswell Park Memorial Institute 1640 medium (RPMI1640) supplemented with 10% FBS and 1% antibiotic-antimycotic) (NC1), MSC medium (NC2), and engineered growth factor cocktail medium for hematopoietic cells (PC). For viability test, apoptosis test, and CD33, CD34 phenotypes test, AML cells were seeded into 96-well plate, each well contained one 3DON/3DMN and each 3DON/3DMN contained 5×10⁴ cells. Specifically, the AML cells in co-culture groups were maintained in 1:1 AML cell line medium:MSC medium as the cell number of AML:MSC were 1:1. The 3DON was differentiated for 21 days before co-culture, 3DMN was formed 3 days before co-culture for the 3DMN to contract and 2DMN was seeded 3 days before co-culture as a fair comparison to the 3DMN. All the groups were seeded with AML cells on the same day. In a 96-well plate, each well was seeded with 5×10⁴ primary AML cells. Trypan blue viability assay on AML cells was performed after three days of co-culture with different niches for the AML cells to interact with the environment. The experiments were performed in triplicates, and a total of three independent experiments were conducted.

### Cd33+ Cd34− Phenotype Analysis by Flow Cytometry

Maintaining the phenotype of AML cells in drug screening platform is necessary to ensure valid screening results. Phenotypes of AML cells were analyzed by flow cytometry. For flow cytometry, each well was seeded with 5×10⁴ primary AML cells. Cells were aspirated after 3 days of co-culture and washed. Cells were stained with allophycocyanin-Cy7 (APC/Cy7) or fluorescein isothiocyanate (FITC) isotype control and monoclonal antibodies (CD33-APC/Cy7 (Abcam), an AML myeloid lineage marker and CD34-FITC (Abcam), a hematopoietic stem cell marker) for 30 minutes on ice. The cells were then washed and resuspended in 5% FBS (200 μL) and passed through 70 μm cell strainer and kept on ice before analysis. The samples were analyzed by LSRFortessa (Bdbiosciences) and ten thousand events per sample were recorded using BD FACSDiva software. Gatings were created to exclude dead cells, debris, and doublets such that only live singlets were analyzed.

### Apoptosis Detection

It is reported that primary AML cells would undergo spontaneous apoptosis rapidly when being cultured ex vivo, therefore, apoptosis detection was performed to analyze whether the in vivo niche could reduce apoptosis [16-18]. Apoptosis was detected using TUNEL assay kit (Abcam). Cells were fixed and stained according to the manufacturer's protocol. In brief, each sample was prepared by pooling primary AML cells from 2 wells (approximately 100000 cells) and were fixed with 1% paraformaldehyde for 15 mins after being cultured for 3 days in different culture conditions. The cells were then washed and transferred to 70% ethanol and incubate for a minimum of 30 minutes at −20° C. Cells were washed again and resuspended in the DNA labelling solution consisting of reaction buffer, TdT enzyme, FITC-dUTP, and ddH₂O and incubated for 1 hour at 37° C. Next, cells were counterstained with Propidium Iodide/rNase A. Cells were analyzed by flow cytometer LSRFortessa (Bdbiosciences). The FITC-labelled TUNEL-positive cells were recognized as apoptotic cells. Gatings were created to exclude debris and doublets such that only single cells were analyzed. Ten thousand events per sample were recorded for analysis using BD FACSDiva software.

### ALP Staining and Von Kossa Staining

ALP staining and von Kossa staining was performed to verify successful osteogenic differentiation of the MSCs in 3DON, using 3DMN as the control without differentiation. The 3DON or 3DMN groups were fixed with 4% paraformaldehyde (PFA) to preserve the sample and cut into 10 μm sections. The samples were stained using alkaline Phosphatase Kit (Sigma) following the manufacturer's protocol with nuclear fast red as counter stain. Von Kossa staining was performed to reveal the calcium deposits. Slides were immersed in 1% silver nitrate (Sigma) solution under ultraviolet for 1 hour and then immersed in 2% sodium thiosulphate (Sigma) solution for 3 minutes to remove unreacted silver according to the previous protocol [101].

### Immunofluorescence Staining

Immunofluorescence staining of the osteogenic markers was performed to verify successful osteogenic differentiation of the MSCs in 3DON. The fixed frozen sections of samples were permeabilized by adding 0.5% Tween-20 for 10 minutes and washed with PBS 3 times, 5 minutes each. Blocking of nonspecific sites was performed by adding 5% of bovine serum albumin (Sigma) for 30 minutes. Primary antibodies osteopontin (ab8448, abcam) and osteocalcin (ab13420, abcam) were diluted in 1:200 concentration and incubated at 4° C. overnight. After washing, secondary antibody was diluted to 1:200 and incubated at room temperature for 1-hour, washed, and mounted with fluorogel II mounting medium with DAPI (Electron microscopy Sciences).

### Live Dead Staining

Live and dead staining was performed on the 3DMN and 3DON to assess the viability of the cells being encapsulated. The live/dead viability kit (L3224, Invitrogen) was used. The samples were washed with PBS thrice and incubated in calcein-AM and ethidium homodimer-1 according to the manufacturer's protocol.

### Scanning Electron Microscopy (SEM)

SEM imaging was performed to observe the structure inside the 3DON. The fabricated 3DONs were rinsed with PBS, then fixed with 4% PFA at 4° C. overnight. They were washed with PBS and followed by serial dehydration (10%, 30%, 50%, 70%, 90%, 95%, and 100%, 15 minutes each). Samples were then undergone critical point drying at the Electron Microscopy Unit (the University of Hong Kong). The internal structure of the coprecipitates was revealed by fracture and sputter-coated by gold. Scanning electron microscopy (SEM) images were captured by Hitachi S-4800. The EDX analysis was performed to map the calcium (Ca) and phosphorus (P) weight content in the samples.

### Sample Drug Screening

For sample drug test, AML cells were cultured in nine different conditions (same as co-culture analysis) in 384 wells for 3 days. For the 3DON/3DMN groups, each well contained one 3DON/3DMN and each 3DON/3DMN contained 5000 cells. For other groups, AML cells of density 5000 cells/well were seeded into each well. On the second day of co-culture, daunorubicin (Sigma) of ten different working concentrations (0-426 UM) and doxorubicin (Sigma) of fifteen different working concentrations (0-2.559 mM) was administered into the culture wells. After 24 hours, AML cells were aspirated into a blank 384 well plate for viability assay using PrestoBlue (Invitrogen) and measured by a fluorescence plate reader (SpectraMax iD3, Molecular Devices) after 3 hours of incubation. The experiments were performed in triplicate, and a total of three independent experiments were conducted.

### Cytokine Array

The cytokine array was used to detect the level of cytokines secreted in different niches. Conditioned medium from 3DON was collected for 3 days post ostoegenic differentiation while that from 3DMN and 2DMN were collected for 3 days post-culture. The collected medium was passed through a 0.2 μm filter and stored at −80° C. Uncultured DMEM-LG full medium was used as a control. The soluble cytokines in the medium were detected by human cytokine antibody array (Abcam). The analysis was performed according to the manufacturer's instructions. Briefly, membranes were blocked in 1× blocking buffer at room temperature for 30 minutes and washed. Undiluted samples were added and incubated overnight at 4° C. and washed. Then, 1× biotin-conjugated anti-cytokines were pipetted onto each membrane and incubated for 2 hours at room temperature and washed. 1×HRP-conjugated streptavidin was then added and incubated for another 2 hours at room temperature and washed. Detection buffer mixture was pipetted onto the membrane and incubated for 2 minutes at room temperature followed by chemiluminescence detection using Azure c300 gel imaging system (Azure Biosystems). Densitometric data were obtained using ImageJ software.

### Statistical Analysis

Statistical calculations were performed with GraphPad Prism 9 (GraphPad Software). The statistical analyses were performed by one-way ANOVA followed by Bonferroni's multiple comparisons test, except for viability test, CD33 CD34 phenotype maintenance, cytokine level comparisons, and IC₅₀ comparisons were analysed by non-parametric one-way ANOVA Kruskal-Wallis test followed by Dunn's multiple comparisons test to reveal the statistically significant differences among different groups with a significance level of 0.05. The IC₅₀ values were determined from the dose-response model built-in to Prism.

### Example 1: Morphological and Histological Analysis of 3DON

### Materials and Methods

The primary AML cells were co-cultured with the 3DON for 3 days in different conditions. Primary AML cells were cultured in three control groups, I: negative control 1 (AML medium) (NC1); II: negative control 2 (MSC medium) (NC2); and III: positive control (engineered growth factor cocktail medium for hematopoietic cells) (PC), and co-cultured in three experimental groups: IV: healthy 2D mesenchymal niche (MSC feeder layer) (2DMN); V: healthy 3D mesenchymal niche (3DMN); VI: healthy 3D osteogenic niche (3DON); VII: AML 2DMN; VIII: AML 3DMN; IX: AML 3DON.

The 3DON was produced by encapsulating MSCs in rat-tail type I collagen. The MSC cell density was 50000 cells/ml, collagen concentration 2 mg/ml, volume of 3DON was 100 μL. The culture medium, IN sodium hydroxide, stock collagen and cell suspension were mixed and pipetted into the wells of a 96-well plate, pre-coated with F127 for an hour at room temperature. Osteogenic differentiation of the encapsulated MSCs were performed 3 days after cell encapsulation. The microspheres were cultured in DMEM-LG supplemented with 10% FBS, 100 nM dexamethasone (Sigma), 50 μM ascorbic acid 2-phosphate (BioChemika), 10 mM b-glycerophosphate (Calbiochem) and 10 ng/ml BMP-2 (PeproTech) as the osteogenic differentiation induction medium. The microspheres were cultured for 21 days with regular medium replenishment every 2-3 days before co-culture.

The 3DMN were produced the same way as 3DON except without the 21 days osteogenic differentiation. The 2DMN group was prepared by seeding 50000 cells in each well of the 96-well plate. The medium used for NC1 was RPMI 1640 medium supplemented with 20% FBS and 1% anti-anti. The medium used for NC2 was DMEM-LG medium supplemented with 10% FBS and, 1% GlutaMAX and 1% anti-anti. For the co-culture groups, the medium was a 1:1 mixed medium of NC1 and NC2 medium.

ALP staining and von Kossa staining was performed to verify successful osteogenic differentiation of the MSCs in 3DON, using 3DMN as the control without differentiation. The 3DON or 3DMN groups were fixed with 4% paraformaldehyde (PFA) to preserve the sample and cut into 10 μm sections. The samples were stained using alkaline Phosphatase Kit (Sigma) following the manufacturer's protocol with nuclear fast red as counter stain. Von Kossa staining was performed to reveal the calcium deposits. Slides were immersed in 1% silver nitrate (Sigma) solution under ultraviolet for 1 hour and then immersed in 2% sodium thiosulphate (Sigma) solution for 3 minutes to remove unreacted silver according to the previous protocol [101].

SEM imaging was performed to observe the structure inside the 3DON. The fabricated 3DONs were rinsed with PBS, then fixed with 4% PFA at 4° C. overnight. They were washed with PBS and followed by serial dehydration (10%, 30%, 50%, 70%, 90%, 95%, and 100%, 15 minutes each). Samples were then undergone critical point drying at the Electron Microscopy Unit (the University of Hong Kong). The internal structure of the coprecipitates was revealed by fracture and sputter-coated by gold. Scanning electron microscopy (SEM) images were captured by Hitachi S-4800. The EDX analysis was performed to map the calcium (Ca) and phosphorus (P) weight content in the samples.

Live and dead staining was performed on the 3DMN and 3DON to assess the viability of the cells being encapsulated. The live/dead viability kit (L3224, Invitrogen) was used. The samples were washed with PBS thrice and incubated in calcein-AM and ethidium homodimer-1 according to the manufacturer's protocol.

### Results

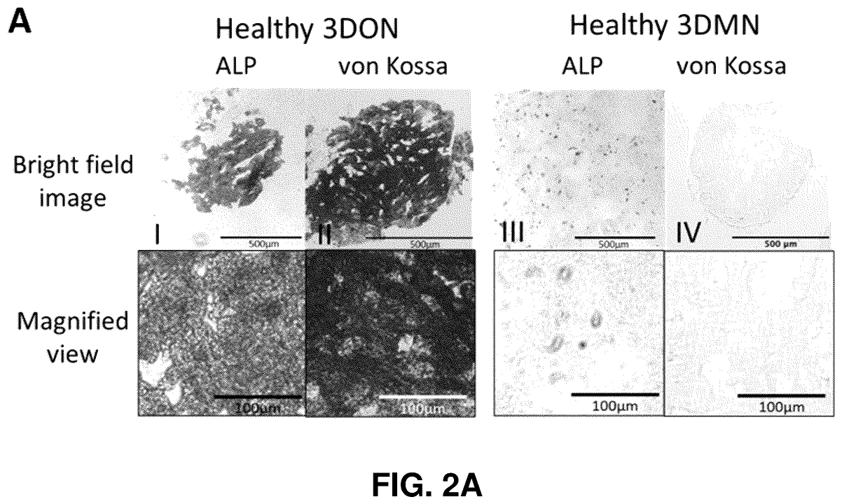

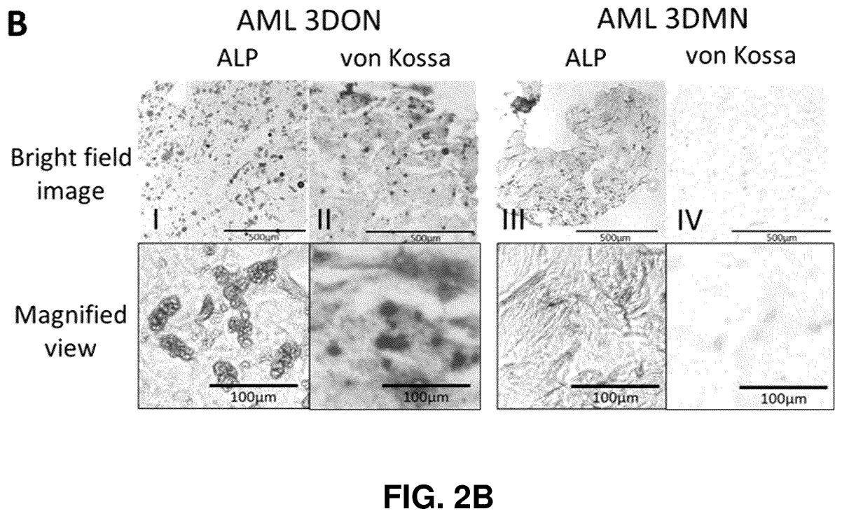

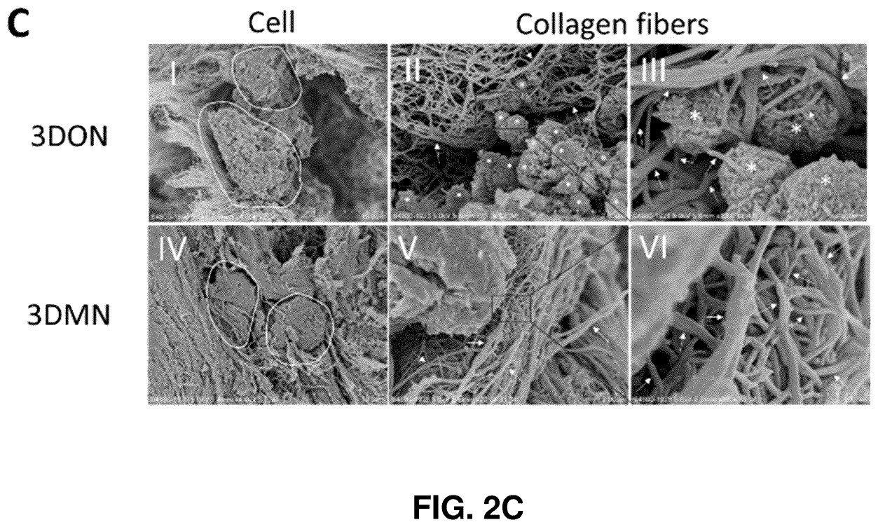

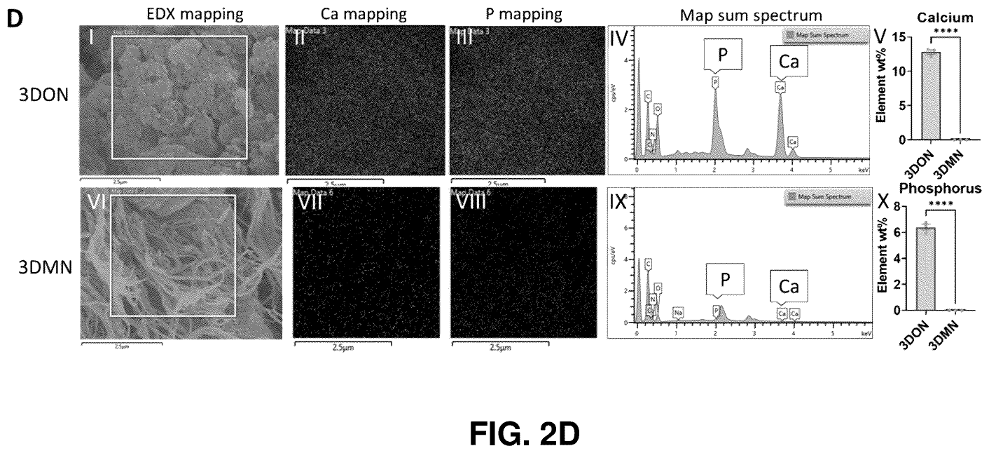

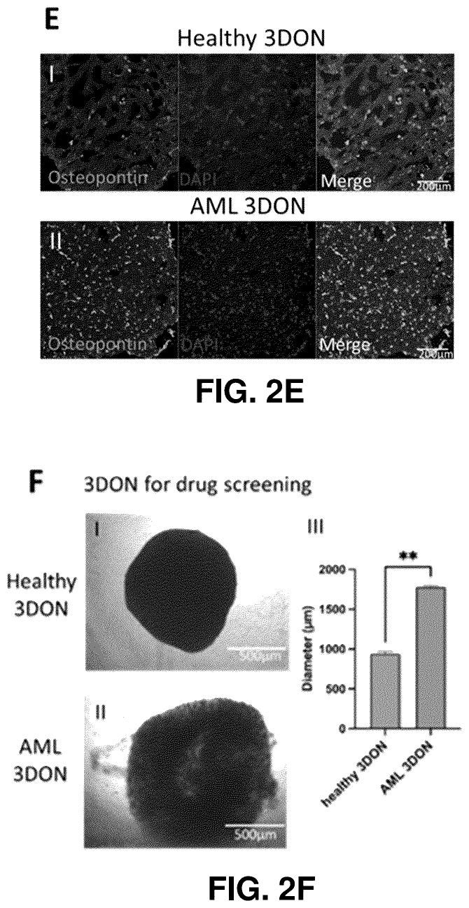

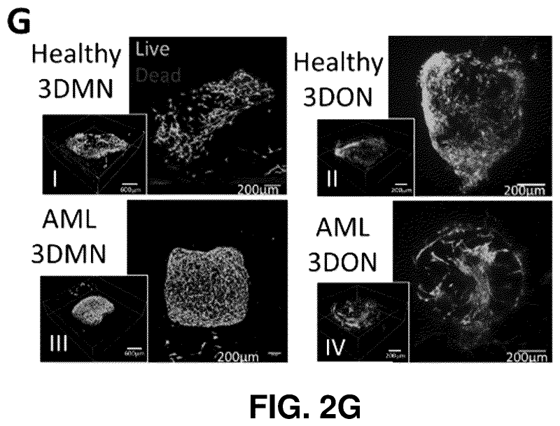

From FIG. 2A, strong positive staining of ALP was shown in healthy 3DON (FIG. 2A(I)) after being osteogenic differentiated for 21 days, compared to the negative staining in 3DMN that was exposed to normal medium (FIG. 2A(III)). In AML MSC, positive staining of ALP was shown in AML 3DON (FIG. 2B(I)), compared to the negative staining in 3DMN (FIG. 2B(III)).

Von Kossa staining reveals calcium deposition as a late-stage osteogenic differentiation marker. After 21 days in osteogenic differentiation (OD) medium, the healthy 3DON were heavily stained black (FIG. 2A(II)), suggesting the presence of calcium phosphate deposits in comparison with the negative staining in healthy 3DMN (FIG. 2A(IV)). Similarly, positive black staining was found on AML 3DON (FIG. 2B(II)) as compared to negative staining in AML 3DMN (FIG. 2B(IV)). Both ALP and von Kossa stainings were not as heavily stained in AML 3DON than in healthy 3DON, suggesting the AML 3DON was not as readily differentiated as that in the healthy 3DON. This result aligned with the previous report on the compromised MSCs osteogenic differentiation capability [35].

Scanning electron microscopy (SEM) revealed the microstructure of 3DON and 3DMN. Low magnification (4 kX) images revealed that the cells (circled) being encapsulated were settling on a collagen fiber meshwork (FIG. 2C(I, IV)). High magnification (20 kX, 100 kX) images showed that calcium granules indicated by asterisks were deposited within the fiber meshwork indicated by arrows in the 3DON during osteogenic differentiation (FIG. 2C(II, III)). For the 3DMN that was undifferentiated, the microstructure contained meshwork of collagen fibers (FIG. 2C(V, VI)). SEM-guided energy-dispersive X-ray spectroscopy (EDX) analysis was performed on the 3DON and 3DMN for analyse the chemical nature of the deposits in FIG. 2D. From FIG. 2D(I), the map sum spectrum identified calcium (13.19%) and phosphorus (6.77%) peaks, demonstrating the 3DON contains calcium (FIG. 2D(III)) and phosphorus (FIG. 2D(IV)), which were elements known to be present during bone formation [36]. In the 3DMN, the map sum spectrum identified 0.01% calcium and 0.00% phosphorus peaks. Unpaired t-test revealed that the calcium level and phosphorus level in 3DON is significantly different from that in 3DMN (p<0.0001).

The presence of osteogenic markers ostcopontin were confirmed by immunofluorescence staining presented in FIG. 2E. Positive staining of osteopontin (FIG. 2E) was observed in healthy and AML 3DON, suggesting that the 3DONs were undergoing osteogenic differentiation.

The bright field images showing the gross appearance of the healthy and AML 3DON in FIG. 2F(I, II), and their diameters were presented in FIG. 2F(III). Mann-Whitney test showed that the diameter of the 3DON during the drug screening tests was significantly different between the healthy group and the AML group (p=0.007). This might be due to the lower contraction ability of MSCs from leukemia patients as compared to those from healthy donors, leading to a lower local cell density, which may explain the decreased osteogenic differentiation efficiency [37].

Live dead staining in FIG. 2G demonstrated that most of the cells were alive in the healthy and AML 3DMN and 3DON. This indicated that the MSCs were viable after being encapsulated in collagen microspheres (FIG. 2G(I, III)) and differentiated osteogenically for 21 days (FIG. 2G(II, IV)).

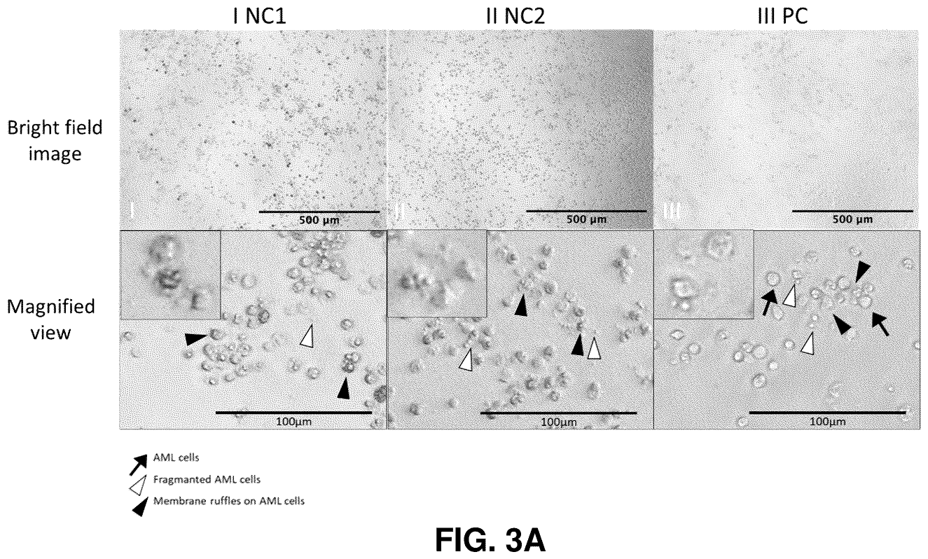

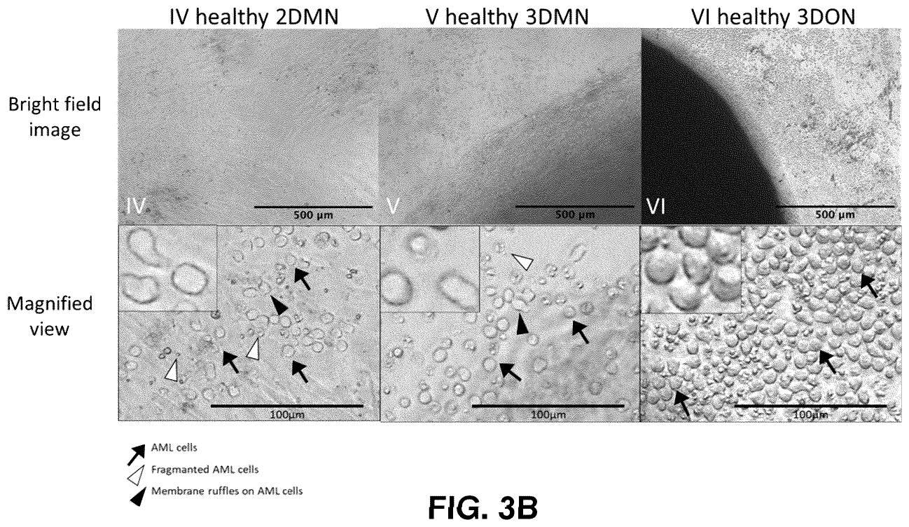

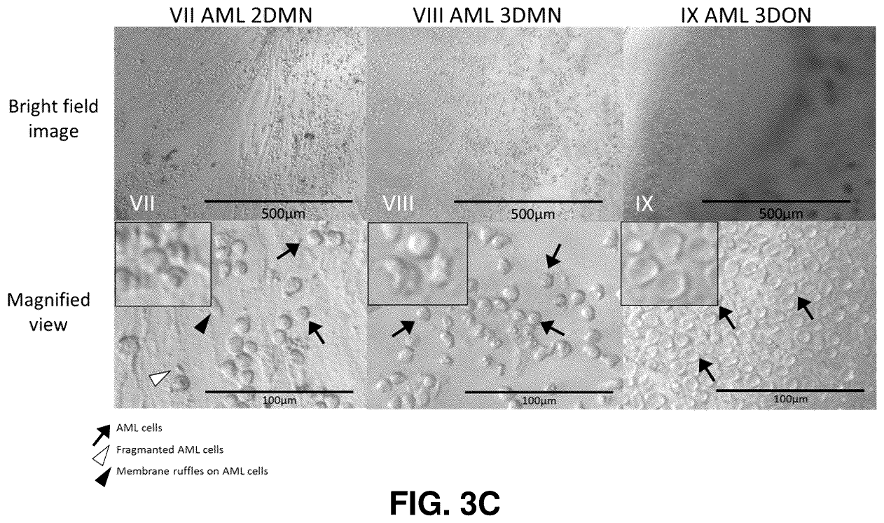

FIG. 3 shows the bright field images of the AML cells being cultured under different conditions on day 2 of co-culture. Both the healthy and AML 3DON became darker while the healthy 3DON became opaque, indicating the presence of calcium deposits, as a result of the osteogenic differentiation of MSCs into osteogenic lineages as previously reported [31], whereas the healthy and AML 3DMN containing undifferentiated MSCs remained less opaque. The AML cells co-cultured with healthy 3DON, AML 2DMN, AML 3DMN, and AML 3DON appeared mostly bright, and rounded (black arrows). From the bright field images, the cell number in healthy 3DON and AML 3DON were much higher than the other groups (NC1, NC2, PC, healthy 2DMN, healthy 3DMN, AML 2DMN, and AML 3DMN) (FIG. 3 (VI, XI). The blasts were in close contact with the 3DON. On the contrary, the other groups (NC1, NC2, PC, healthy 2DMN, healthy 3DMN) had a much lower cell number and most of the cells became shrunk or fragmented (white triangles) (FIG. 3 (I-V)). Ruffling of the plasma membrane was also observed in some of the cells in NC1, NC2, PC, healthy 2DMN and healthy 3DMN groups (black triangles) (FIG. 3 (I-V)), suggesting the cells might be undergoing apoptosis. Rounded AML blasts were found in PC, healthy 2DMN, and healthy 3DMN groups but the number was much lower than the 3DON group.

### Example 2: Viability of AML Cells was Greatly Enhanced Upon Co-Culture with 3DON

### Materials and Methods

To analyze the viability of AML cells upon co-culture with different groups, trypan blue exclusion test was conducted after three days of co-culture. The preparation of all the culture groups were described in example 1. Trypan blue counting was performed by aliquoting 10 μL sample of cell suspension from each well for cell counting.

Apoptosis was detected using TUNEL assay kit (Abcam). Cells were fixed and stained according to manufacturer's protocol. In brief, each sample was prepared by pooling primary AML cells from 2 wells (total approximately 100000 cells), and were fixed with 1% paraformaldehyde for 15 mins after being cultured for 3 days in different culture conditions. The cells were then washed and transferred to 70% ethanol. Cells were washed and resuspended in the DNA labelling solution that consist of reaction buffer, TdT enzyme, FITC-dUTP, and ddH₂O and incubated for 1 hour at 37° C. Next, cells were counterstained with Propidium Iodide/RNase A. Cells were analyzed by flow cytometer LSRFortessa (Bdbiosciences). The FITC-labelled TUNEL-positive cells were recognized as apoptotic cells. Ten thousand events per sample were recorded using BD FACSDiva software.

### Results

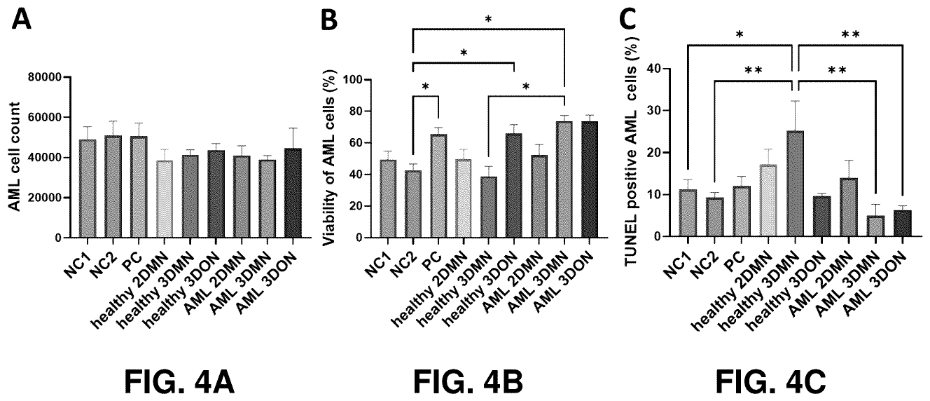

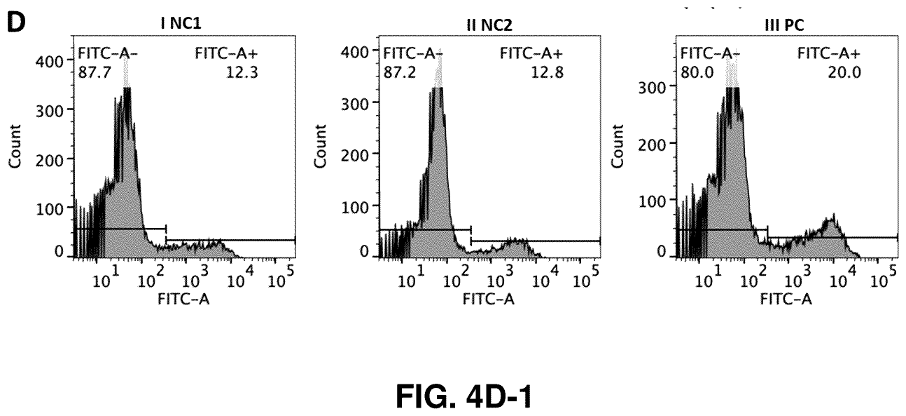

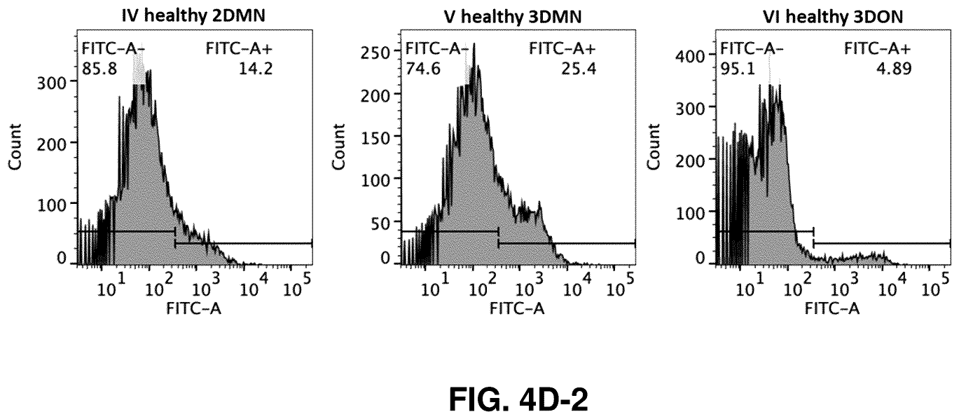

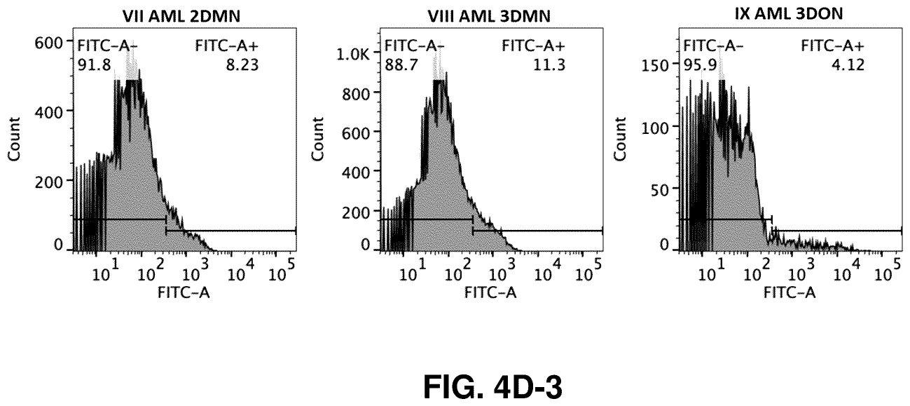

FIG. 4A showed the AML cell number in different groups. There was no significant difference in cell number among all the groups. One-way ANOVA test showed no significant (n.s.) difference in all groups (p=0.804). The AML cell counts were about 39000-51000.

For viability, as FIG. 4B shows, the NC groups (AML and MSC medium) and the undifferentiated healthy MSCs (2DMN and 3DMN groups) had only on average, 49%, 43%, 49% and 39% viable cells respectively while AML 2DMN had 52% viable cells. However, the group of healthy 3DON and PC gave better results, they both had 66% viable cells. Viabilities of AML blasts cultured with AML 3DMN and AML 3DON niches were the highest among all groups, both reaching 74% viability, suggesting that these native AML niches indeed support AML viability and that explains why AML cells away from their native leukemic niche, will die or become apoptotic. Kruskal Wallis (p<0.0001) with Dunn's post-hoc test showed that AML 3DMN (p=0.0383), healthy 3DON (p=0.0384) and PC (p=0.0480) were significantly higher than NC2, and AML 3DMN was significantly higher than healthy 3DMN (p=0.0437).

To quantify the population of apoptotic cells, fragmented DNA was stained by terminal deoxynucleotidyl transferase dUTP nick end labeling (TUNEL) assay, and FIG. 4C shows the percentage of apoptotic cells in each group and FIG. 4D shows the representative flow cytometry plots of TUNEL assay. Blasts cultured in healthy undifferentiated MSCs 3DMN and 2DMN had the highest population of TUNEL positive cells at 25% and 17% respectively. Followed by AML 2DMN 14%, PC 12%, NC1 11%, healthy 3DON 9.7% and NC2 9.3%. The groups with the lowest number of apoptotic cells were AML 3DMN and AML 3DON with 5.1% and 6.3% of the total population respectively. One-way ANOVA (p=0.0017) with Bonferroni's post-hoc test showed that the percentage of apoptosis cells in healthy 3DMN was significantly higher than NC2 (p=0.006), AML 3DMN (p=0.0014), 3DON (p=0.0038), and NC1 (p=0.0373).

### Example 3: Cytokines Secreted by 3DON were Pro-Tumoral

### Materials and Methods

A cytokine array screening of 80 cytokines and growth factors was performed to identify the type and quantity of secreted proteins by the experimental groups, against the NC2 group (uncultured DMEM-LG medium) and the PC group (uncultured engineered growth factor cocktail medium).

The preparation of 3DON were described in example 1. Conditioned medium from 3DON was collected for 3 days post ostoegenic differentiation while that from 3DMN and 2DMN were collected for 3 days post-culture. The collected medium was passed through a 0.2 μm filter and stored at −80° C. Uncultured DMEM-LG full medium was used as a control. The soluble cytokines in the medium were detected by human cytokine antibody array (Abcam). The analysis was performed according to the manufacturer's instructions. Briefly, membranes were blocked in 1× blocking buffer at room temperature for 30 minutes and washed. Undiluted samples were added and incubated overnight at 4° C. and washed. Then, 1× biotin-conjugated anti-cytokines were pipetted onto each membrane and incubated for 2 hours at room temperature and washed. 1×HRP-conjugated streptavidin was then added and incubated for another 2 hours at room temperature and washed. Detection buffer mixture was pipetted onto the membrane and incubated for 2 minutes at room temperature followed by chemiluminescence detection using Azure c300 gel imaging system (Azure Biosystems). Densitometric data were obtained using ImageJ software.

### Results

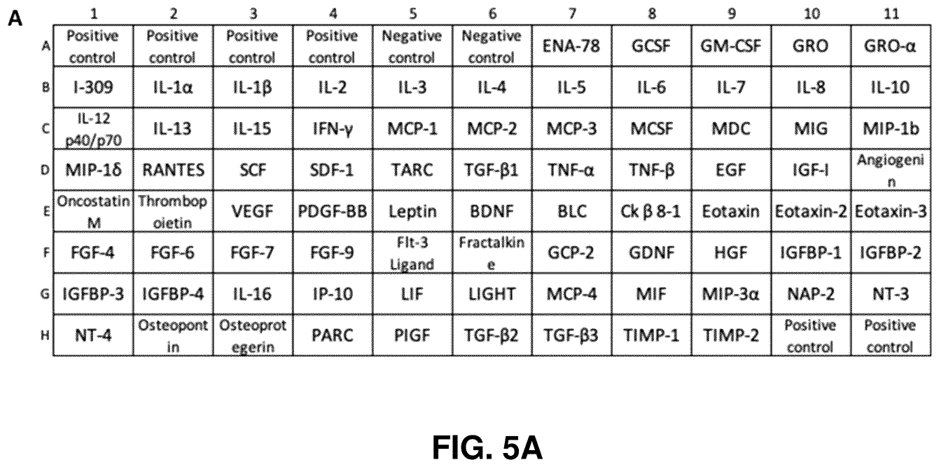

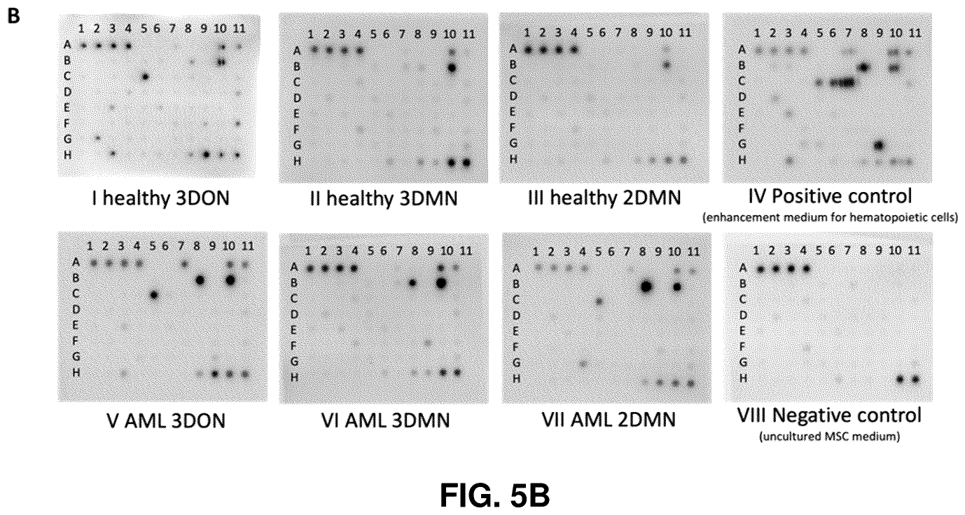

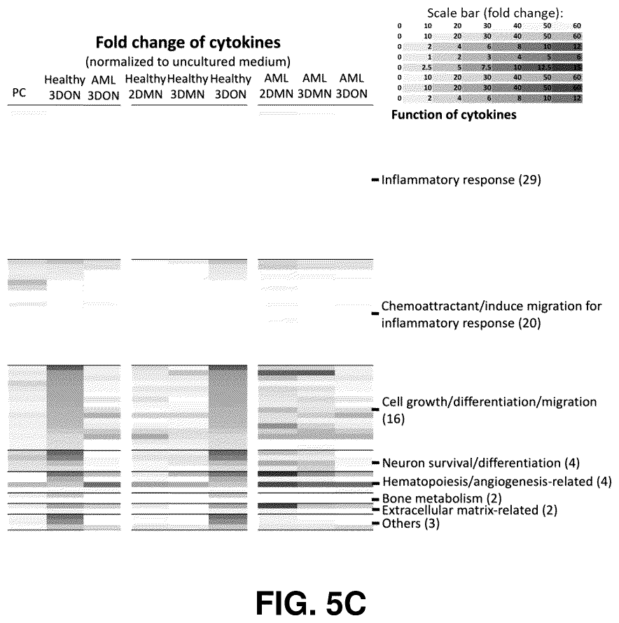

FIG. 5A shows schematic representation of the cytokine spot positions. The images of representative cytokine arrays, including conditioned medium from healthy and AML 3DON, 3DMN, 2DMN, PC, and uncultured MSC medium were presented in FIG. 5B-5C shows the heatmaps on the fold changes of cytokines in groups of 3 for comparison purpose: (I) PC, healthy 3DON, and AML 3DON; (II) healthy 2DMN, healthy 3DMN, and healthy 3DON; (III) AML 2DMN, AML 3DMN, and AML 3DON, against the control group NC1. The cytokines were categorized according to their major functions.

The 10 proteins with the highest fold changes in healthy 3DON were interleukin-8 (IL-8) (37.94), osteoprotegerin (OPG) (35.48), tissue inhibitor of metalloproteinases-2 (TIMP-2) (22.77), monocyte chemoattractant protein-1 (MCP-1) (18.87), interleukin-10 (IL-10) (17.43), growth-related oncogene-α (GRO-α) (16.58), interleukin-6 (IL-6) (15.00), growth-related oncogene (GRO) (14.18), interleukin-7 (IL-7) (12.84), and angiogenin (10.61). In AML 3DON, the 10 highest fold changes were MCP-1 (22.52), IL-8 (21.17), TIMP-2 (18.69), IL-6 (18.35), vascular endothelial growth factor (VEGF) (11.34), epithelial neutrophil-activating peptide 78 (ENA-78) (8.18), GRO (6.25), angiogenin (5.97), macrophage inflammatory protein-3α (MIP-3α) (5.69) and GRO-α (5.65). In AML 3DMN, the 10 proteins with the highest fold changes were IL-6 (46.79), MCP-1 (24.3), TIMP-2 (20.63), IL-8 (19.51), VEGF (11.48), hepatocyte growth factor (HGF) (9.93), Angiogenin (9.01), GRO (8.15), TIMP-1 (6.94), GRO-α (5.96) (Table 1).

TABLE 1
Fold changes in cytokine levels in healthy (2DMN,
3DMN, 3DON) and AML (2DMN, 3DMN, 3DON).

| | | | healthy | healthy | healthy | AML | AML | AML |
|---|---|---|---|---|---|---|---|---|
| Cytokines | | PC | 2DMN | 3DMN | 3DON | 2DMN | 3DMN | 3DON |
| ENA-78 | D | 14.37 | 1.02 | 1.46 | 5.16 | 7.88 | 5.28 | 8.18 |
| GCSF | C | 4.99 | 0.63 | 0.64 | 5.84 | 3.69 | 2.06 | 1.04 |
| GM-CSF | C | 5.73 | 0.30 | 0.44 | 8.52 | 1.44 | 0.93 | 0.59 |
| GRO | D | 16.61 | 2.07 | 6.95 | 14.18 | 8.28 | 8.15 | 6.25 |
| GRO-α | D | 13.53 | 0.37 | 1.83 | 16.58 | 6.65 | 5.96 | 5.65 |
| I-309 | D | 1.47 | 0.96 | 1.62 | 1.06 | 1.47 | 0.85 | 1.54 |
| IL-1α | C | 3.17 | 1.07 | 1.17 | 1.40 | 1.17 | 0.83 | 1.24 |
| IL-1β | C | 2.73 | 0.93 | 1.26 | 1.87 | 1.18 | 0.97 | 1.11 |
| IL-2 | C | 1.79 | 1.03 | 1.49 | 3.15 | 1.15 | 1.12 | 1.20 |
| IL-3 | C | 2.17 | 0.86 | 1.28 | 4.00 | 1.29 | 1.22 | 1.10 |
| IL-4 | C | 3.29 | 1.12 | 1.08 | 5.66 | 1.44 | 1.19 | 1.21 |
| IL-5 | C | 7.05 | 0.36 | 0.87 | 6.08 | 1.32 | 1.13 | 0.70 |
| IL-6 | C | 46.94 | 1.36 | 2.92 | 15.00 | 73.83 | 46.79 | 18.35 |
| IL-7 | C | 12.20 | 0.50 | 0.48 | 12.84 | 3.36 | 1.83 | 1.09 |
| IL-8 | D | 27.94 | 6.02 | 15.02 | 37.94 | 27.95 | 19.51 | 21.17 |
| IL-10 | C | 23.06 | 0.48 | 0.87 | 17.43 | 2.59 | 1.87 | 2.13 |
| IL-12 p40/p70 | C | 1.29 | 0.99 | 1.01 | 1.19 | 1.19 | 0.62 | 1.18 |
| IL-13 | C | 1.57 | 1.08 | 0.93 | 1.31 | 0.88 | 0.58 | 1.01 |
| IL-15 | C | 1.41 | 1.23 | 1.04 | 1.78 | 1.11 | 0.82 | 1.31 |
| IFN-γ | C | 1.71 | 0.75 | 1.12 | 2.76 | 0.77 | 1.00 | 0.82 |
| MCP-1 | D | 17.36 | 1.15 | 1.31 | 18.87 | 32.69 | 24.30 | 22.52 |
| MCP-2 | C | 32.16 | 1.02 | 1.01 | 6.24 | 1.88 | 1.48 | 2.10 |
| MCP-3 | D | 42.34 | 1.01 | 0.89 | 6.62 | 1.94 | 1.34 | 1.32 |
| MCSF | C | 6.08 | 0.99 | 0.87 | 6.23 | 4.90 | 2.83 | 1.61 |
| MDC | C | 2.49 | 0.90 | 0.76 | 6.16 | 2.48 | 1.48 | 1.24 |
| MIG | D | 2.42 | 0.98 | 1.93 | 6.56 | 3.39 | 2.07 | 2.35 |
| MIP-1b | C | 5.66 | 0.83 | 1.02 | 5.36 | 2.38 | 1.83 | 1.52 |
| MIP-1δ | C | 1.45 | 1.67 | 1.55 | 1.84 | 1.32 | 1.33 | 2.61 |
| RANTES | D | 4.3 | 1.13 | 1.33 | 1.25 | 1.31 | 1.46 | 1.01 |
| SCF | A | 1.83 | 1.99 | 1.65 | 2.20 | 1.50 | 1.60 | 2.27 |
| SDF-1 | 0 | 1.47 | 1.66 | 1.63 | 3.73 | 1.48 | 1.50 | 1.98 |
| TARC | C | 1.62 | 2.09 | 1.60 | 2.16 | 2.24 | 1.82 | 1.76 |
| TGF-β1 | E | 3.00 | 0.96 | 1.02 | 5.74 | 1.06 | 0.91 | 1.06 |
| TNF-α | C | 2.70 | 0.89 | 0.83 | 3.75 | 1.04 | 0.77 | 0.83 |
| TNF-β | C | 1.86 | 0.88 | 0.80 | 4.78 | 1.58 | 1.05 | 0.95 |
| EGF | E | 1.35 | 0.75 | 0.88 | 4.62 | 2.20 | 1.16 | 1.23 |
| IGF-I | E | 1.20 | 0.80 | 1.01 | 4.80 | 4.26 | 2.10 | 2.39 |
| Angiogenin | A | 1.98 | 3.18 | 6.35 | 10.61 | 15.06 | 9.01 | 5.97 |
| Oncostatin M | C | 1.10 | 1.55 | 1.69 | 1.84 | 0.64 | 1.54 | 1.91 |
| Thrombopoietin | C | 1.52 | 3.05 | 3.56 | 2.44 | 0.42 | 0.97 | 1.14 |
| VEGF | A | 6.32 | 7.90 | 7.32 | 5.46 | 13.65 | 11.48 | 11.34 |
| PDGF-BB | E | 1.82 | 3.19 | 3.23 | 4.70 | 2.39 | 3.41 | 4.94 |
| Leptin | H | 1.34 | 2.26 | 2.01 | 3.57 | 2.17 | 2.08 | 2.81 |
| BDNF | F | 2.11 | 1.42 | 1.46 | 3.84 | 1.10 | 1.18 | 0.99 |
| BLC | D | 1.45 | 1.37 | 1.26 | 3.98 | 1.17 | 1.09 | 0.70 |
| Ck β 8-1 | D | 1.12 | 1.09 | 1.01 | 4.26 | 1.50 | 1.06 | 0.76 |
| Eotaxin | D | 1.07 | 1.24 | 1.24 | 2.87 | 2.23 | 1.30 | 1.11 |
| Eotaxin-2 | D | 1.00 | 1.02 | 1.28 | 2.74 | 3.66 | 1.63 | 1.54 |
| Eotaxin-3 | D | 1.50 | 0.86 | 2.01 | 4.62 | 8.13 | 3.57 | 3.36 |
| FGF-4 | E | 1.16 | 2.87 | 2.43 | 3.49 | 3.26 | 4.55 | 3.52 |
| FGF-6 | E | 1.28 | 5.33 | 2.25 | 2.64 | 5.13 | 4.90 | 4.94 |
| FGF-7 | E | 1.55 | 2.26 | 0.70 | 5.24 | 1.87 | 2.71 | 1.93 |
| FGF-9 | E | 1.16 | 1.07 | 1.16 | 2.35 | 0.66 | 0.87 | 0.46 |
| Flt-3 Ligand | A | 1.55 | 2.18 | 1.55 | 6.91 | 2.59 | 2.45 | 1.46 |
| Fractalkine | D | 1.34 | 1.58 | 1.64 | 4.73 | 1.31 | 1.67 | 0.73 |
| GCP-2 | E | 1.91 | 1.83 | 1.86 | 5.51 | 2.83 | 2.31 | 1.05 |
| GDNF | F | 1.65 | 1.16 | 1.08 | 4.42 | 2.19 | 1.62 | 0.56 |
| HGF | E | 1.86 | 1.79 | 3.91 | 6.25 | 9.60 | 9.93 | 1.40 |
| IGFBP-1 | E | 1.45 | 1.23 | 0.99 | 4.38 | 6.80 | 2.94 | 1.60 |
| IGFBP-2 | E | 1.47 | 1.71 | 1.71 | 6.01 | 4.00 | 2.22 | 1.89 |
| IGFBP-3 | E | 1.13 | 1.15 | 0.96 | 5.12 | 1.56 | 1.85 | 0.70 |
| IGFBP-4 | E | 1.26 | 2.40 | 0.87 | 9.62 | 3.07 | 2.96 | 1.46 |
| IL-16 | D | 1.48 | 1.57 | 0.68 | 5.12 | 2.32 | 2.36 | 0.99 |
| IP-10 | D | 2.81 | 1.40 | 1.04 | 3.51 | 7.23 | 4.42 | 1.75 |
| LIF | C | 3.18 | 1.39 | 1.16 | 3.61 | 1.93 | 1.71 | 0.67 |
| LIGHT | C | 1.30 | 1.31 | 1.38 | 4.23 | 2.04 | 1.69 | 0.76 |
| MCP-4 | D | 1.28 | 1.23 | 1.04 | 5.26 | 2.18 | 1.52 | 0.37 |
| MIF | C | 2.05 | 1.42 | 1.13 | 5.20 | 3.58 | 2.48 | 0.80 |
| MIP-3α | D | 25.76 | 1.89 | 0.82 | 6.56 | 10.20 | 5.28 | 5.69 |
| NAP-2 | D | 2.38 | 1.01 | 0.90 | 3.39 | 4.74 | 3.10 | 1.30 |
| NT-3 | F | 1.50 | 0.94 | 1.30 | 2.36 | 2.74 | 2.23 | 0.91 |
| NT-4 | F | 1.34 | 0.91 | 0.72 | 1.59 | 1.20 | 1.74 | 0.86 |
| Osteopontin | B | 1.58 | 1.55 | 0.77 | 2.35 | 1.84 | 2.44 | 1.19 |
| Osteoprotegerin | B | 13.70 | 2.22 | 1.05 | 35.48 | 3.87 | 2.12 | 3.68 |
| PARC | H | 1.76 | 1.32 | 0.91 | 6.23 | 1.70 | 1.52 | 1.02 |
| PIGF | H | 1.57 | 1.45 | 1.08 | 7.48 | 1.70 | 1.21 | 0.80 |
| TGF-β2 | E | 1.03 | 1.77 | 1.49 | 2.60 | 0.57 | 0.59 | 0.16 |
| TGF-β3 | E | 1.42 | 1.26 | 1.26 | 5.48 | 2.27 | 1.40 | 0.66 |
| TIMP-1 | G | 4.59 | 4.30 | 5.37 | 9.35 | 9.14 | 6.94 | 5.42 |
| TIMP-2 | G | 10.75 | 11.23 | 6.67 | 27.77 | 55.65 | 20.63 | 18.69 |

‡—A: Hematopoiesis/angiogenesis-related; B: Bone metabolism; C: Inflammatory response; D: Chemoattractant/induce migration for inflammatory response; E: Cell growth, differentiation or migration; F: Neuron survival/differentiation; G: Extracellular matrix-related; H: Others.

In healthy 3DON, there were 64 out of the total 80 in the cytokine array that had a higher fold change compared to PC and most of them had functions related to cell growth, differentiation or migration (FIG. 5C). Among them, the cytokines that had the greatest difference in fold change were OPG (21.78), TIMP-2 (17.02), IL-8 (9.99), angiogenin (8.62), insulin like growth factor binding protein-4 (IGFBP-4) (8.36), placental growth factor (PIGF) (5.91), Fms Related Receptor Tyrosine Kinase 3-ligand (Flt-3 ligand) (5.36), tissue inhibitor of metalloproteinases-1 (TIMP-1) (4.75), insulin-like growth factor binding protein-4 (IGFBP-2) (4.53) and pulmonary and activation-regulated chemokine (PARC) (4.47) (Table 2).

TABLE 2
Fold changes in cytokine levels in healthy (2DMN, 3DMN,
3DON) and AML (2DMN, 3DMN, 3DON) compared to PC.

| | | healthy | healthy | healthy | AML | AML | AML |
|---|---|---|---|---|---|---|---|
| | | 2DMN - | 3DMN - | 3DON - | 2DMN - | 3DMN - | 3DON - |
| Cytokines | ‡ | PC | PC | PC | PC | PC | PC |
| ENA-78 | D | −13.35 | −12.91 | −9.21 | −6.49 | −9.09 | −6.20 |
| GCSF | C | −4.36 | −4.35 | 0.86 | −1.30 | −2.93 | −3.95 |
| GM-CSF | C | −5.43 | −5.29 | 2.80 | −4.29 | −4.80 | −5.13 |
| GRO | D | −14.55 | −9.66 | −2.44 | −8.33 | −8.46 | −10.37 |
| GRO-α | D | −13.15 | −11.70 | 3.05 | −6.88 | −7.57 | −7.87 |
| I-309 | D | −0.51 | 0.15 | −0.41 | 0.00 | −0.62 | 0.07 |
| IL-1α | C | −2.10 | −2.00 | −1.77 | −2.01 | −2.35 | −1.93 |
| IL-1β | C | 1.79 | −1.46 | −0.85 | −1.54 | −1.76 | −1.62 |
| IL-2 | C | −0.75 | −0.30 | 1.36 | −0.64 | −0.66 | 0.59 |
| IL-3 | C | −1.31 | −0.89 | 1.84 | −0.87 | −0.95 | −1.06 |
| IL-4 | C | −2.18 | 2.21 | 2.37 | −1.85 | −2.10 | −2.08 |
| IL-5 | C | −6.69 | −6.18 | −0.97 | −5.72 | −5.92 | −6.34 |
| IL-6 | C | −45.58 | −44.02 | −31.94 | 26.88 | −0.15 | −28.59 |
| IL-7 | C | −11.70 | −11.72 | 0.64 | −8.84 | −10.37 | −11.12 |
| IL-8 | D | −21.92 | 12.92 | 10.00 | 0.01 | −8.43 | −6.77 |
| IL-10 | C | −22.58 | −22.20 | −5.63 | −20.47 | −21.19 | −20.93 |
| IL-12 p40/p70 | C | −0.31 | −0.28 | −0.10 | −0.10 | −0.67 | 0.11 |
| IL-13 | C | −0.49 | 0.64 | −0.26 | −0.69 | −0.99 | −0.57 |
| IL-15 | C | −0.18 | −0.37 | 0.37 | −0.30 | −0.59 | −0.10 |
| IFN-γ | C | −0.96 | −0.60 | 1.05 | −0.94 | −0.71 | −0.89 |
| MCP-1 | D | −16.21 | −16.05 | 1.52 | 15.33 | 6.94 | 5.17 |
| MCP-2 | C | −31.14 | −31.15 | −25.91 | −30.27 | −30.67 | −30.06 |
| MCP-3 | D | −41.33 | −41.45 | −35.73 | −40.40 | −41.00 | −41.03 |
| MCSF | C | −5.09 | −5.20 | 0.16 | −1.17 | −3.24 | −4.47 |
| MDC | C | −1.60 | 1.73 | 3.67 | −0.02 | −1.01 | −1.26 |
| MIG | D | −1.44 | 0.49 | 4.14 | 0.97 | −0.35 | −0.08 |
| MIP-1b | C | −4.83 | −4.64 | −0.30 | −3.28 | −3.83 | −4.14 |
| MIP-1δ | C | 0.21 | 0.10 | 0.39 | −0.13 | −0.12 | 1.16 |
| RANTES | D | −3.18 | −2.98 | −3.06 | −3.00 | −2.85 | −3.30 |
| SCF | A | 0.15 | −0.18 | 0.37 | −0.33 | 0.23 | 0.43 |
| SDF-1 | C | 0.20 | 0.17 | 2.27 | 0.01 | 0.04 | 0.51 |
| TARC | C | 0.47 | −0.02 | 0.54 | 0.62 | 0.20 | 0.15 |
| TGF-β1 | E | −2.04 | −1.98 | 2.74 | −1.94 | −2.09 | −1.94 |
| TNF-α | C | −1.81 | −1.87 | 1.05 | −1.66 | −1.93 | −1.87 |
| TNF-β | C | −0.98 | 1.06 | 2.92 | −0.28 | −0.81 | −0.91 |
| EGF | E | −0.60 | −0.46 | 3.27 | 0.85 | −0.19 | −0.12 |
| IGF-I | E | −0.39 | −0.18 | 3.61 | 3.06 | 0.90 | 1.20 |
| Angiogenin | A | 1.20 | 4.37 | 8.63 | 13.07 | 7.03 | 3.99 |
| Oncostatin M | C | 0.45 | 0.59 | 0.75 | −0.46 | 0.44 | 0.81 |
| Thrombopoietin | C | 1.53 | 2.04 | 0.92 | 1.09 | −0.55 | −0.38 |
| VEGF | A | 1.59 | 1.00 | 0.86 | 7.33 | 5.17 | 5.02 |
| PDGF-BB | E | 1.37 | 1.41 | 2.89 | 0.57 | 1.59 | 3.12 |
| Leptin | H | 0.92 | 0.67 | 2.24 | 0.83 | 0.74 | 1.47 |
| BDNF | F | −0.69 | −0.65 | 1.73 | −1.01 | −0.93 | −1.12 |
| BLC | D | −0.08 | −0.19 | 2.53 | −0.29 | −0.36 | −0.75 |
| Ck β 8-1 | D | −0.03 | −0.11 | 3.14 | 0.38 | −0.06 | −0.36 |
| Eotaxin | D | 0.17 | 0.16 | 1.80 | 1.16 | 0.23 | 0.04 |
| Eotaxin-2 | D | 0.01 | 0.27 | 1.74 | 2.66 | 0.63 | 0.53 |
| Eotaxin-3 | D | −0.64 | 0.52 | 3.13 | 6.64 | 2.08 | 1.87 |
| FGF-4 | E | 1.71 | 1.27 | 2.32 | 2.10 | 3.38 | 2.36 |
| FGF-6 | E | 4.05 | 0.98 | 1.36 | 3.86 | 3.63 | 3.66 |
| FGF-7 | E | 0.71 | −0.85 | 3.69 | 0.32 | 1.17 | 0.38 |
| FGF-9 | E | −0.09 | 0.00 | 1.19 | −0.50 | −0.29 | −0.70 |
| Flt-3 Ligand | A | 0.64 | 0.00 | 5.36 | 1.04 | 0.91 | −0.09 |
| Fractalkine | D | 0.24 | 0.31 | 3.39 | −0.02 | 0.33 | −0.60 |
| GCP-2 | E | −0.08 | 0.04 | 3.60 | 0.92 | 0.40 | −0.86 |
| GDNF | F | −0.49 | −0.57 | 2.77 | 0.54 | −0.03 | −1.09 |
| HGF | E | −0.07 | 2.05 | 4.38 | 7.74 | 8.06 | −0.47 |
| IGFBP-1 | E | −0.22 | −0.46 | 2.93 | 5.34 | 1.49 | 0.14 |
| IGFBP-2 | E | 0.24 | 0.23 | 4.54 | 2.53 | 0.75 | 0.42 |
| IGFBP-3 | E | 0.02 | 0.16 | 3.99 | 0.43 | 0.73 | −0.43 |
| IGFBP-4 | E | 1.14 | −0.39 | 8.37 | 1.82 | 1.70 | 0.21 |
| IL-16 | D | 0.09 | −0.80 | 3.64 | 0.83 | 0.88 | −0.49 |
| IP-10 | D | −1.41 | −1.76 | 0.70 | 4.42 | 1.61 | 1.05 |
| LIF | C | −1.79 | −2.02 | 0.43 | 1.25 | −1.47 | −2.51 |
| LIGHT | C | 0.01 | 0.08 | 2.93 | 0.74 | 0.39 | −0.54 |
| MCP-4 | D | −0.05 | −0.24 | 3.98 | 0.90 | 0.24 | −0.91 |
| MIF | C | −0.63 | −0.92 | 3.15 | 1.53 | 0.44 | 1.24 |
| MIP-3α | D | −23.87 | −24.94 | −19.20 | −15.57 | −20.48 | −20.07 |
| NAP-2 | D | −1.37 | −1.48 | 1.01 | 2.36 | 0.72 | −1.08 |
| NT-3 | F | −0.57 | −0.21 | 0.86 | 1.24 | 0.73 | −0.59 |
| NT-4 | F | −0.43 | −0.63 | 0.24 | −0.14 | 0.40 | −0.49 |
| Osteopontin | | −0.03 | −0.82 | 0.77 | 0.25 | 0.86 | −0.39 |
| Osteoprotegerin | | −11.49 | −12.65 | 21.78 | −9.83 | −11.58 | −10.03 |
| PARC | H | −0.44 | −0.84 | 4.47 | −0.05 | −0.24 | −0.74 |
| PIGF | H | −0.12 | −0.48 | 5.91 | 0.13 | −0.35 | −0.76 |
| TGF-β2 | E | 0.74 | 0.46 | 1.57 | −0.46 | −0.44 | −0.87 |
| TGF-β3 | E | −0.16 | −0.15 | 4.06 | 0.86 | −0.02 | −0.75 |
| TIMP-1 | G | −0.30 | 0.78 | 4.75 | 4.54 | 2.35 | 0.82 |
| TIMP-2 | G | 0.48 | −4.08 | 17.03 | 44.90 | 9.88 | 7.94 |

‡—A: Hematopoiesis/angiogenesis-related; B: Bone metabolism; C: Inflammatory response; D: Chemoattractant/induce migration for inflammatory response; E: Cell growth, differentiation or migration; F: Neuron survival/differentiation; G: Extracellular matrix-related; H: Others.

In AML 3DON, which best supports AML growth, there were 23 cytokines that had a higher fold change compared to PC and were mostly related to cell growth, differentiation or migration and also hematopoiesis or angiogenesis-related. (FIG. 5C). Among them, the cytokines that had the greatest difference in fold change were TIMP-2 (7.94), VEGF (5.02), Angiogenin (3.99), fibroblast growth factor-6 (FGF-6) (3.66), PDGF-BB (3.12), fibroblast growth factor-4 (FGF-4) (2.36), eotaxin-3 (1.87), leptin (1.47), insulin-like growth factor-1 (IGF-1) (1.20) and macrophage inflammatory protein-1δ (MIP-1δ) (1.16) (FIG. 11).

In AML 3DMN, there were 34 cytokines that had a higher fold change compared to PC and were mostly related to cell growth, differentiation or migration and also hematopoiesis or angiogenesis-related (FIG. 5C). The cytokines that had the greatest difference in fold change among them were TIMP-2 (9.88), HGF (8.06), angiogenin (7.03), MCP-1 (6.94), VEGF (5.17), FGF-6 (3.63), FGF-7 (3.38), TIMP-1 (2.35), Eotaxin-3 (2.08), PDGF-BB (1.59). The cytokines secreted by the AML niches (AML 3DON and AML 3DMN) revealed that the AML niches may support tumor growth and progression, as the secreted cytokines that were released in the highest amount have major functions related to cell growth, migration and angiogenesis.

The PC group was prepared by pooling the medium from three engineered murine fibroblast cell lines, M210B4+JGCSF+JIL3 HyTK, sl/sl SCF+IL3 HyTK, and sl/sl FLT3L irradiated at 80 Gy, mixed in 1:1:1 ratio for culture. The medium collected after 3 days was the engineered growth factor cocktail medium. The highest fold changes in PC group as compared to the NC2 group (MSC medium) were IL-6 (46.94), MCP-3 (42.34), MCP-2 (32.15), IL-8 (27.93), MIP-3α (25.76), IL-10 (23.06), MCP-1 (17.35), GRO (16.61), ENA-78 (14.37), and OPG (13.70). These cytokines all belong to inflammatory responses except for OPG which is for bone metabolism.

Most of the cytokines that had a lower fold change in AML 3DON and healthy 3DON compared to PC were related to the inflammatory response (FIG. 5C), which had limited pro-tumoral effect, suggesting that 3DON may be more effective than PC in maintaining the survival of AML cells. For the growth-related proteins (green), the healthy 3DON group showed high concentration and all were higher than the PC group, in AML 3DON, half of them were higher than PC (FIG. 5C), which may be the primary reason why AML do not survive well in culture due to the lack of native BM niches particularly the osteogenic niche. Therefore, AML survived better in 3DON which better mimics the native BM microenvironment.

In addition, the engineered cell lines were designed to produce SCF, G-CSF, IL-3, and FLT-3 ligand, and among the detected levels of these four proteins in the medium (1.83-fold, 4.98-fold, 2.16-fold and 1.54-fold respectively), SCF was lower than that released by AML 3DON (2.27-fold), and all were lower than that released by healthy 3DON (2.20-fold, 5.84-fold, 4.00-fold, and 6.90-fold respectively), which may be the reason why the healthy and AML 3DON are better than the PC in supporting the survival of AML cells in vitro.

Besides, the 3DON and 3DMN niche groups, but not the PC group, also involved contact-mode interactions with the AML cells during co-cultures. Research has found that the protective effect on leukemia cells was reduced in a culture system lack of cell-cell contact [39]. AML-collagen interactions such as through integrin α2β1 binding [40], AML-MSC or AML-osteoblast interactions by integrin α4β1 binding would reduce the apoptosis of AML cells, which may be another explanation of the increased viability in the 3DON and 3DMN groups compared to the PC group.

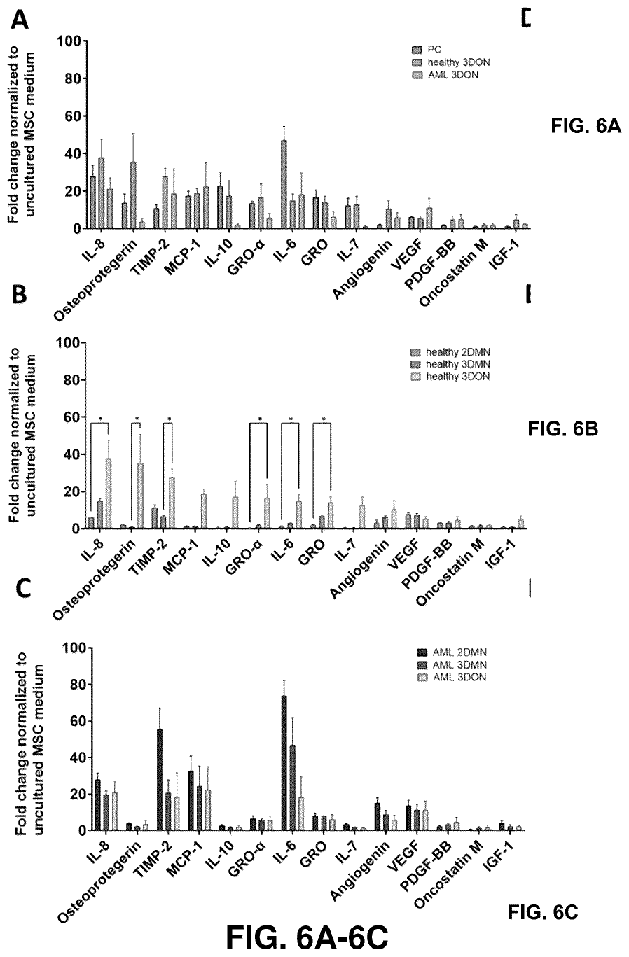

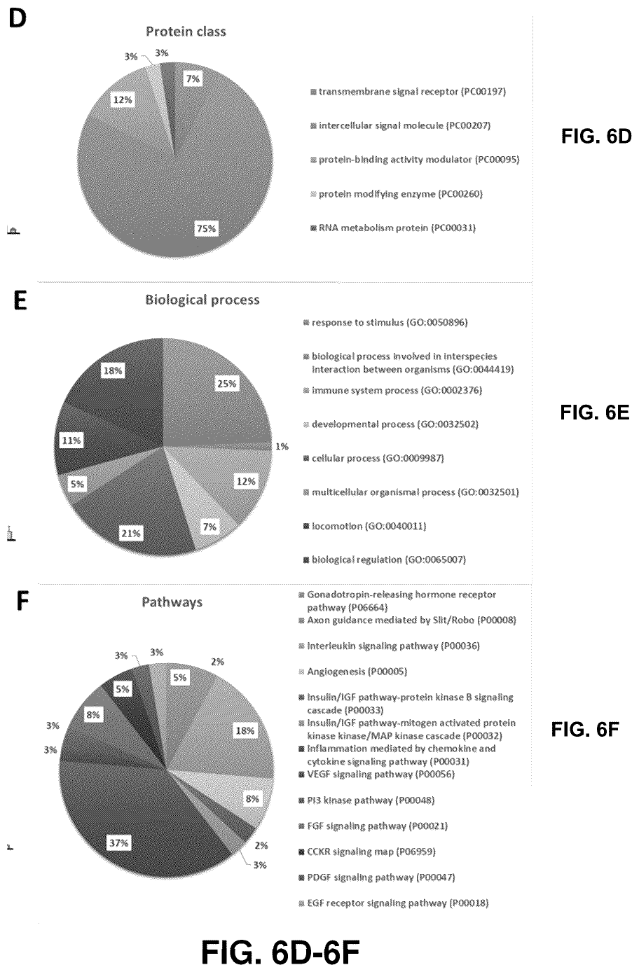

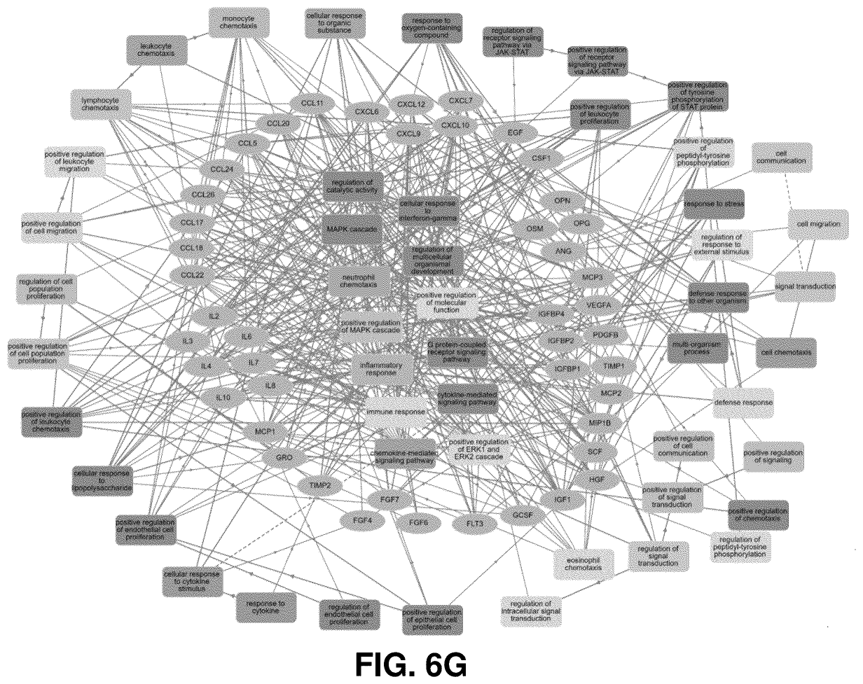

FIG. 6A-6G showed the fold changes of the cytokines that had the highest intensities or had pro-tumoral effects in different groups for comparison. FIG. 6A compared the secretion among PC, healthy 3DON, and AML 3DON. For MCP-1, VEGF, angiogenin and IGF-1, AML 3DON had a greater fold change than PC and healthy 3DON. PC had a greater fold change in IL-6 and GRO compared to healthy 3DON and AML 3DON.

FIG. 6B compared the fold changes of cytokines secreted by healthy 2DMN, healthy 3DMN, and healthy 3DON. Nearly all cytokines were secreted in healthy 3DON in greater amounts. Kruskal Wallis (p=0.0036) with Dunn's post-hoc test showed that there were significant differences between healthy 2DMN and healthy 3DON for IL-8 (p=0.0219), GRO-α (p=0.0219), IL-6 (p=0.0219) and GRO (p=0.0219). Significant differences were found between healthy 3DMN and healthy 3DON for OPG (p=0.0219) and TIMP-2 (p=0.0219). This indicated that the secretion of IL-8, GRO-α, IL-6 and GRO would be greatly enhanced in a 3D culture environment that was osteogenically differentiated.

The fold changes of cytokines detected in the conditioned medium of AML 2DMN, AML 3DMN, and AML 3DON were shown in FIG. 6C. Unexpectedly, for AML MSC, the quantity of most of the cytokines, including pro-tumoral cytokines in AML 2DMN were greater than that in AML 3DMN or AML 3DON. However, the AML cells' survival was lower in AML 2DMN than other co-culture groups. One possible reason was the secretion of considerably large amount of IL-6, which is an inflammation-associated cytokine in AML 2DMN (73-fold change) as compared to AML 3DON (18-fold change), AML 3DMN (47-fold change), and healthy 3DON (15-fold change). It was reported that IL-6 enhances fas-mediated extrinsic apoptosis in different cancers including leukemia [70]. It was suggested that this effect was due to the upregulation of death receptor 5 through the activation of p53 [70], probably explaining the lower viability observed in AML 2DMN.

To further understand how the cytokines present in the PC, healthy 3DON, AML 3DMN, and AML 3DON groups aided the survival of the AML cells, protein annotation through evolutionary relationship gene ontology-slim (PANTHER GO-Slim) analysis was performed. For protein class, the enriched cytokines were mostly belonged to intercellular signal molecules (75%) and protein-binding activity modulators (12%) (FIG. 6D). They were also involved in a number of pathways, including inflammation mediated by chemokine and cytokine signaling pathway (37%), interleukin signaling pathway (18%), FGF signaling (8%) and angiogenesis (8%) (FIG. 6F). For biological process, most of these cytokines were related to response to stimulus (25%), cellular process (21%), biological regulation (18%) and immune system process (12%) (FIG. 6E). The increase in cytokines related to FGF signaling pathway may suggest that these niches supported the survival of AML cells through regulating the FGF pathway, which were known to promote fundamental biological processes including survival, proliferation, differentiation and motility [220].

Gene ontology term enrichment analysis (FIG. 6G) of the upregulated cytokines in the groups that supported the survival of AML cells the best (PC, healthy 3DON, AML 3DMN, and AML 3DON groups) compared to NC2 revealed several pathways that may be relevant to the survival of AML cells. These include the positive regulation of mitogen-activated protein kinase (MAPK) cascade, extracellular signal-regulated kinases (ERK) 1 and ERK2 cascade, and Janus kinase (JAK)-signal transducer and activator of transcription (STAT) pathway.

Only 80 cytokines were assessed in this experiment, there could be more proteins released by the 3DON that were pro-tumoral but not covered by the cytokine chip here. For instance, a study demonstrated that AML cells in fact induce osteogenic differentiation in bone marrow-derived MSCs by secreting BMP-2 to activate Smad1/5 phosphorylation and connective tissue growth factor (CTGF) expression which in turn enhances AML engraftment [14]. It has been demonstrated that BMP-2 was found to be present in the healthy 3DON, therefore BMP-2 may be another factor that associates with in the survival of AML but not included in the cytokine chip [31], Systemic proteomics study is therefore needed in the future to analyze the secretome from the 3DON thoroughly.

### Example 4: Phenotype of AML Cells was Maintained in Co-Culture Environment

### Materials and Methods

The preparation of all the culture groups were described in example 1. To check for any changes in phenotype, cells were aspirated after 3 days of co-culture and washed. Cells were stained with allophycocyanin-Cy7 (APC/Cy7) or fluorescein isothiocyanate (FITC) isotype control and monoclonal antibodies (CD33-APC/Cy7 (Abcam) and CD34-FITC (Abcam)) for 30 minutes on ice. The cells were then washed and resuspended in 200 μl 5% FBS and passed through 70 μm cell strainer and kept on ice before analysis. The samples were analyzed by LSRFortessa (Bdbiosciences) and ten thousand events per sample were recorded using BD FACSDiva software.

### Results

To check whether the phenotype of AML cells were maintained in the BM organoid, immunophenotyping by flow cytometry was performed by staining two leukemia associated immunophenotypes (LAIPs), CD33 and CD34. (Kern et al. 2010) The cut-off was defined as follows: “positive” (≥40%), “dimly positive” (20-40%) and “negative” (<20%). (Zeijlemaker, Gratama, and Schuurhuis 2014).

CD33 is a myeloid marker while CD34 is a hematopoietic stem cell marker. According to the French-American-British classification (FAB), myeloid cell differentiation were categorized into seven stages, M0-M7. (Plesa et al. 2008) Stages having both CD33+ and CD34+ cells could be myeloid progenitor cells (M0), monoblast (M4), or immature megakaryoblast (M7). Stages with CD33+ and CD34− cells are myeloid monocytic progenitor cells (M1), myeloblast (M2), promyelocyte (M3), or promonocyte (M5). No stage has CD33− and CD34+ cells. (Plesa et al. 2008) The patient samples used for this experiment were clinically classified as FAB stages M1 and M5. Cell markers involved for both of the stages therefore should be CD33 positive and CD34 negative (CD33+CD34−). (Casasnovas et al. 2003; Khalidi et al. 1998; Plesa et al. 2008).

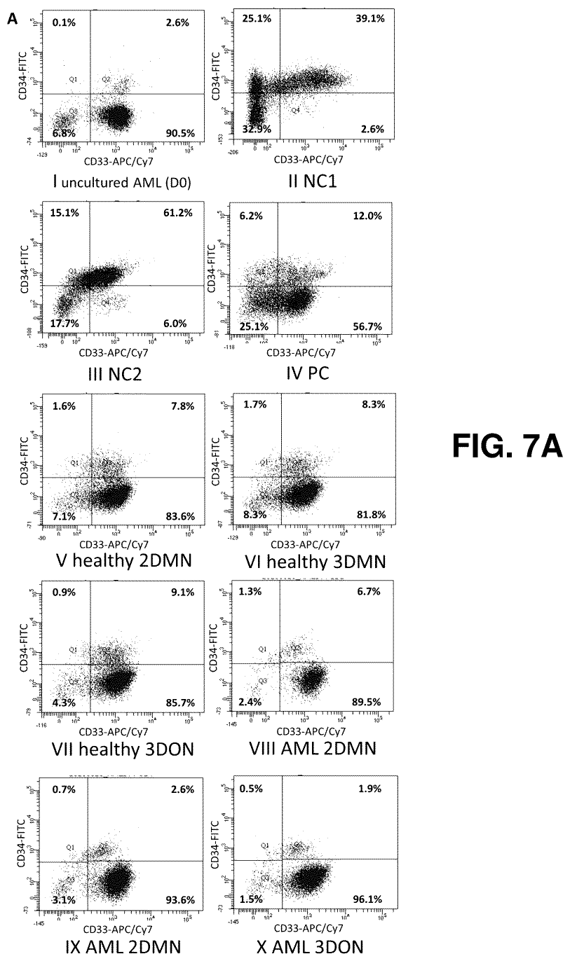

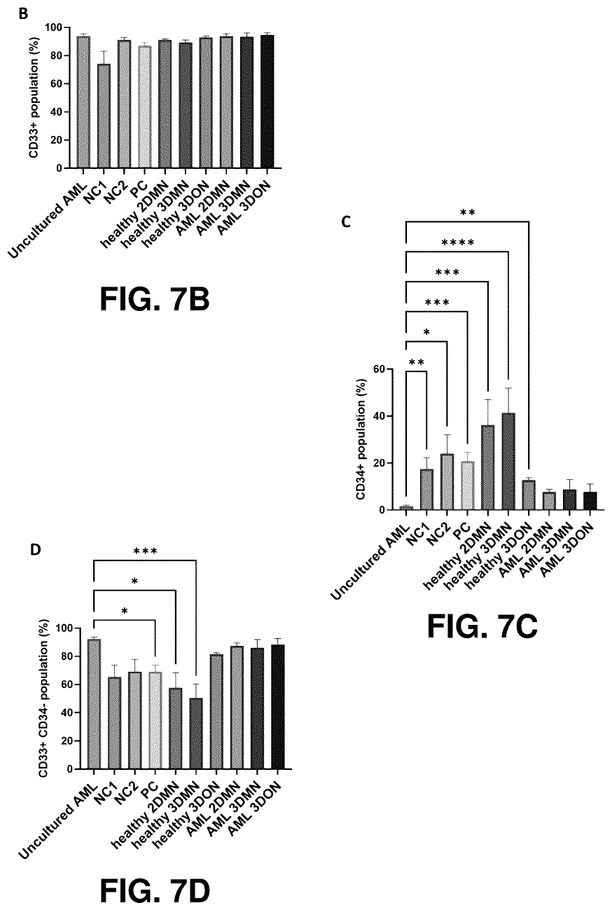

Representative flow cytometry plots were presented in FIG. 7A. Cells on day 0 that did not undergo 3-day culture were used at a control (DO control). From the flow cytometry results (FIG. 7B), CD33 was found to be positive in all the groups, except for NC1, reaching more than 85% of the total cell population. Healthy 3DON, AML 2DMN, AML 3DMN, and AML 3DON were the groups that had the highest population of CD33+ with 92.9%, 93.7%, 93.7% and 94.6% respectively. AML cells cultured in NC2, PC, healthy 2DMN, and healthy 3DMN groups had a slightly lower population of 87-91% cells with CD33+ marker compared to AML 3DON. NC1 had the lowest CD33+ population of 74%.

FIG. 7C showed that CD34 was found positive in healthy 3DMN, and dimly positive in healthy 2DMN, NC2 and PC. It was negative in NC1, healthy 3DON, AML 2DMN, AML 3DMN, AML 3DON, and DO control. DO control, AML 2DMN, and AML 3DON had the lowest population of CD34 positive cells, which were 1.66%, 7.73%, and 7.73% respectively and were regarded as negative. AML 3DMN, healthy 3DON, and NC1 had slightly higher CD34+ population of 8.89%, 12.7%, and 17.5% respectively, which were also regarded as negative. NC2, PC and healthy 2DMN had dimly positive CD34+ signals with population percentages between 20-40%. AML cells co-culture with the healthy 3DMN group resulted in the highest CD34+ population of 41%, which was regarded as positive. Kruskal Wallis test (p<0.0001) with Dunn's post-hoc test showed that the D0 control was significantly lower than NC1 (p=0.0075), NC2 (p=0.0109), PC (p=0.0002), healthy 2DMN (p=0.0001), healthy 3DMN (p<0.0001), and healthy 3DON (p=0.0076). Among them, AML 2DMN and AML 3DON preserved the CD34-phenotype of the AML blasts the best with 7.73%.

FIG. 7D showed that the groups that had CD33+ and CD34-cell populations for more than 80% were healthy 3DON, AML 2DMN, AML 3DMN, AML 3DON, and uncultured AML (DO control), with 81.6%, 87.4%, 86.2%, 88.4% and 92.3% respectively. Other groups had less than 70% of the cell population that had CD33+ and CD34−. NC1, NC2, and PC had a population that was slightly lower at 65.3%, 69.3% and 69.1%. Healthy 2DMN and healthy 3DMN had the lowest CD33+CD34−populations of 57.7% and 50.5. Kruskal Wallis test (p=0.0002) with Dunn's post-hoc test showed that the CD33+CD34-population in D0 control was significantly higher than PC (p=0.0175), healthy 2DMN (p=0.0112), and healthy 3DMN (p=0.0008). Among them, AML 3DON preserved the CD33+CD34-phenotype of the AML blasts the best with 88.4% which was closest to the D0 control of 92.2%, further supporting the fact that AML niche better supports the AML blasts.

### Example 5: AML Cells were More Sensitive to Daunorubicin Upon Co-Culture with 3DON

### Materials and Methods

For drug screening, AML cells were cultured in six different conditions. The preparation of all the culture groups were described in example 1 except that the 384 well-plate was used instead of 96-well plate, and also AML cells of density 5000 cells/well and 3DON of 10 μl were seeded into each well. On the second day of co-culture, daunorubicin (Sigma) of ten different working concentrations (0-320 μM) was administered into the culture wells. After 24 hours, AML cells were aspirated into a blank 384 well plate for viability assay using PrestoBlue (Invitrogen) and measured by a fluorescence plate reader (SpectraMax iD3, Molecular Devices) after 3 hours of incubation. The IC₅₀ was calculated as a measure of drug potency in different groups. Daunorubicin, which is one of the drugs being used as a standard treatment clinically, was used as the sample drug (Bertoli et al. 2017).

### Results

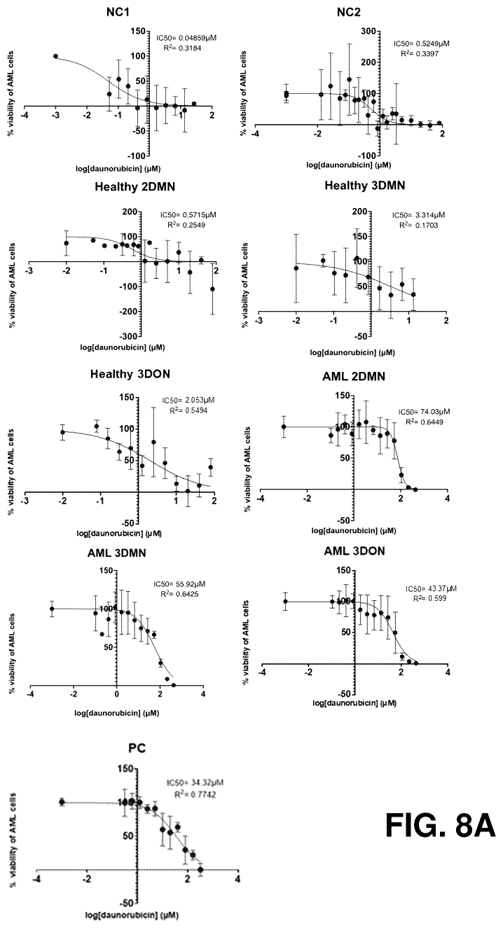

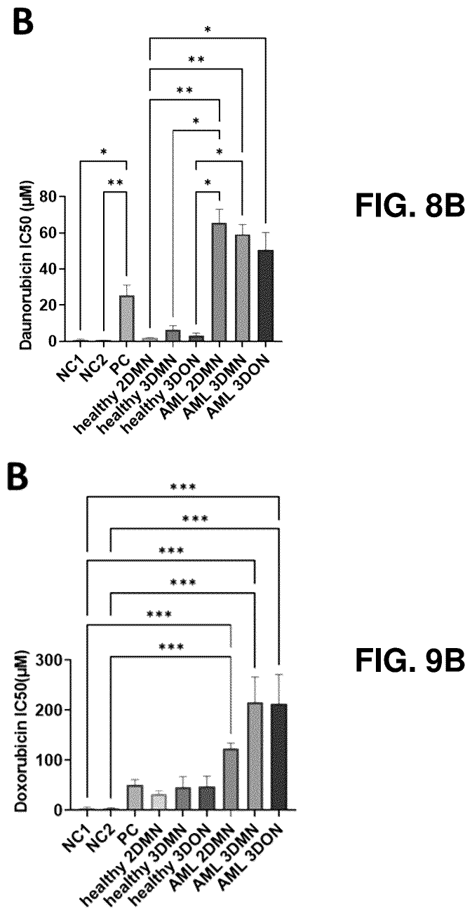

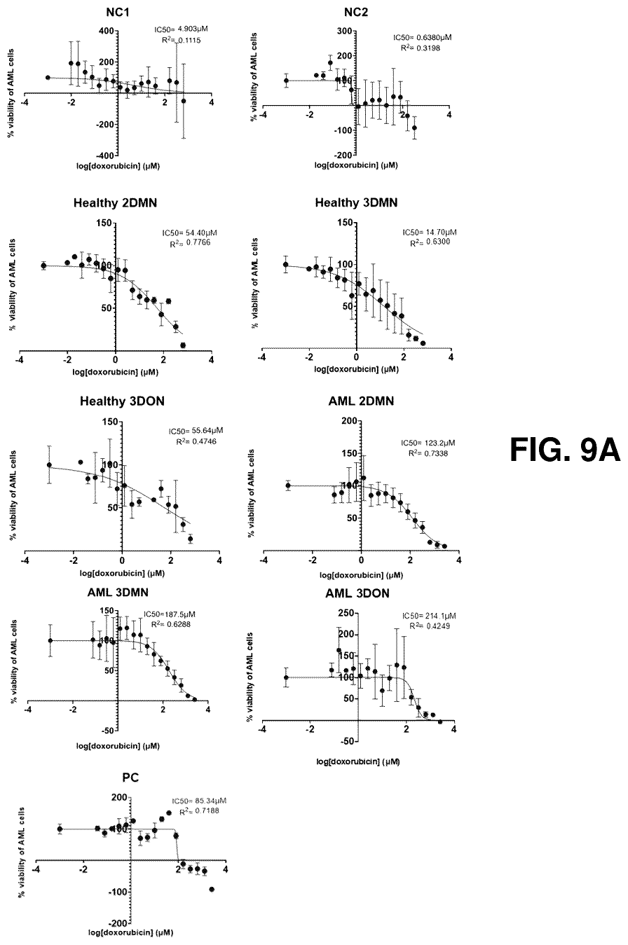

FIG. 8A showed the dose-dependent response curves of primary AML cells towards daunorubicin under different culture conditions while FIG. 8B summarized the IC₅₀ values of different groups. The IC₅₀s of all the AML niche groups were much higher in values than that of all the healthy niche groups. The IC₅₀ of the groups with the AML MSCs derived niche groups, including AML 2DMN, AML 3DMN, and AML 3DON, were the highest among all the groups, they were 74.0 μm, 55.9 μm, and 43.4 μM, respectively, and showed statistically significantly difference with NC1 and NC2 by Kruskal Wallis test (p<0.0001) with Dunn's post-hoc test. The IC₅₀ of NC1 was significantly lower than AML 2DMN (p=0.0007), AML 3DMN (p=0.0025), AML 3DON (p=0.0052), NC2 was significantly lower than AML 2DMN (p=0.0002), AML 3DMN (p=0.0008) and AML 3DON (p=0.0016). The IC₅₀ of the PC group was slightly lower, which was 34.3 μM. Kruskal Wallis test (p<0.0001) with Dunn's post-hoc test revealed that NC2 was significantly lower than PC (p=0.0206). The IC₅₀ of the healthy niches groups were much lower than that of the AML niches group, specifically, they were 0.571 μM, 3.31 μm, and 2.05 μm for healthy 2DMN, healthy 3DMN and healthy 3DON, respectively and these values were similarly low with no significant difference among themselves, but healthy 2DMN was statistically significantly lower than AML 2DMN (p=0.0266) from Kruskal Wallis test (p<0.0001) with Dunn's post-hoc test. The IC₅₀ of the negative controls and the healthy MSC co-cultures were even lower, NC1 was 0.0486 μm, NC2 was 0.525 μM, which could be due to the non-optimal culture condition, causing poorer viability of AML cells.

### Example 6: Development and Testing of 3D Osteogenic Niche (3DON)

Acute myeloid leukemia (AML) is one of the most prevalent acute leukaemia [1]. It is the most lethal hematopoietic cancer with a 5-year survival rate of around 27% [2,3]. AML has over 24 genetically defined subtypes [4], making it a very heterogeneous disease and hence causing poor prognosis [5,6]. Currently, the standard treatment for AML is a 7+3 regimen (7 days of cytarabine with 3 days of anthracycline such as daunorubicin or doxorubicin) but it only results in 50% overall survival [7]. This is followed by consolidation chemotherapy and hematopoietic stem cell transplantation for some high-risk patients [8]. Relapse caused by chemoresistant leukemic cells occurs in more than 70% of patients [9]. Aged individuals, who are the majority of AML patients, may not tolerate the intensive chemotherapy or respond to the treatments due to their higher risk of complex karyotype and TP53 mutation [5]. With the recent advancement in AML pharmacotherapies, tailor-made treatment for patients, especially the elderly, could be made possible by personalized medicine, with potential benefits such as less side effects and improvement in treatment outcomes.

Bone marrow (BM) is a viscous tissue within the bone cavity responsible for haematopoiesis [10]. The two dynamic bone marrow niches-osteogenic niche (ON) that are made up of osteoblasts, osteocytes, and osteoclasts; and the perivascular niche, which consists of endothelial cells and megakaryocytes, interact to regulate the functions of haematopoietic stem cells (HSCs) [11]. Mesenchymal stromal cell (MSC) is an important cell type as they establish the hematopoietic environment by differentiating into many essential components of the BM niche, and they also affect the proliferation, differentiation, and homing of HSCs [12]. Leukemia cells are genetically mutated from HSCs [13]. In fact, leukemic cells interact with the BM microenvironment by different mechanisms, for example they would activate bone morphogenetic protein 2 (BMP-2) and Smad1/5 signaling in MSCs to induce osteogenic differentiation to build their own favorable niche for supporting leukemogenesis [12,14].

AML cells have prolonged survival in vivo [15]. However, primary AML cells rapidly undergo spontaneous or stress-induced apoptosis in ex vivo cultures, possibly owing to the change in the microenvironment, albeit this phenomenon is highly patient-dependent due to heterogeneity [16-18]. As a result, AML cells in conventional cultures can hardly be used to screen for drugs and therapies, because cell death may not be attributable to the drug being screened and evaluated. This discrepancy between the in vitro and the in vivo behavior of AML cells, in particular, the spontaneous apoptosis and the poor survival of the AML cells in cultures may be attributable to the lack of the pro-tumoral BM microenvironment consisting of soluble factors such as growth factors, soluble adhesion molecules and cell-cell/cell-extracellular matrix (ECM) interactions in native cancer tissues.

Different methods have been developed to improve ex vivo culture of primary AML cells [18], including the development of engineered conditioned medium with stem cell factor (SCF), granulocyte colony-stimulating factor (G-CSF), interleukin-3 (IL-3) [17,20]; liquid tumor models such as 2D co-cultures with MSCs or endothelial cells to mimic the BM niche by secreting cytokines [21,22]; and 3D co-cultures such as MSC-coated ceramic scaffolds [23], collagen I or fibronectin coated polylactic-co-glycolic acid (PLGA) and polyurethane (PU) scaffolds [24], polyethylene glycol (PEG) hydrogels with HUVEC and MSCs [25]. However, the preparation of the engineered conditioned medium is tedious, time-consuming, costly, and inefficient because viral transfection of three different cell lines to over-express three different cytokines is necessary for producing the required medium [26]. Furthermore, the engineered conditioned medium and the 2D co-culture environment failed in mimicking the cell-cell or cell-ECM contacts, whereas the existing 3D cultures in a wide range of materials, although being more physiologically relevant, they are yet to recapitulate the pro-tumoral osteogenic niche [18]. In addition, studies have found AML-derived MSCs are different from healthy donors' MSCs in terms of differentiation ability and hematopoiesis support ability [27] [28], however, a leukemia-derived osteogenic niche 3D model is lacking for AML studies.

To address the inadequacy of the existing AML drug screening platforms, reconstituting biomimetic 3D osteogenic niche (3DON) for AML culture is warranted. Progress is underway in developing a 3D microencapsulation platform using naturally occurring extracellular matrix to fabricate physiologically relevant and ECM-based 3D microtissues including those derived from stem cells such as chondrogenic and osteogenic organoids, mature cells such as nucleus pulposus cells and cancer cells such as neuroblastoma cells [33]. Here, it was hypothesized that reconstituting a leukemic 3DON through BM MSC-derived osteogenic organoids, will support the survival and phenotype maintenance of primary AML cells upon co-culture, therefore providing a physiologically relevant in vitro model for personalized chemotherapy drug screening. Moreover, MSCs derived from both healthy individuals and AML patients will be used for forming the bone marrow niches to investigate their differences in supporting AML survival for drug screening applications.

FIG. 1A-1B shows a schematic diagram of the overall experimental design. Specifically, primary AML cells were cultured in three control groups, I: negative control 1 (AML medium) (NC1); II: negative control 2 (MSC medium) (NC2); and III: positive control (engineered growth factor cocktail medium for hematopoietic cells) (PC) niches: IV: healthy 2D mesenchymal niche (MSC feeder layer) (healthy 2DMN); V: healthy 3D mesenchymal niche (healthy 3DMN); VI: healthy 3D osteogenic niche (healthy 3DON), as well as three AML experimental groups where MSCs from AML donors were used in engineering the bone marrow niches: VII: AML 2DMN; VIII: AML 3DMN; IX: AML 3DON. The NC groups were used to confirm that the differences between the co-culture groups and the NC groups were indeed due to the effect of the co-culture but not the effect of the medium. The positive control has been reported to significantly reduce apoptosis of AML cells [20,26] as it contained conditioned medium from engineered murine stromal cell lines that produce stem cell factor (SCF), granulocyte colony-stimulating factor (G-CSF), interleukin-3 (IL-3) and fms-like tyrosine kinase 3 ligand (FLT3 ligand) [20,26]. AML medium and MSC medium were the negative controls because of the absence of stromal cells, ECM and supplementation of cytokines. Human MSCs from healthy and AML donors were used to recapitulate the BM niche to support AML cultures in the six co-culture experimental groups. 2DMN was prepared by seeding MSCs as a 2D feeder layer. 3DMN was prepared by encapsulating MSCs in collagen. 3DON was prepared by differentiating the 3DMN into the osteogenic lineage. The primary AML cells were cultured under different conditions and collected for subsequent evaluation of their viability, apoptosis, phenotype maintenance, and drug response. Medium from the co-culture groups and PC before co-culture were also collected for cytokine secretion microarray assay.

Results demonstrated that the reconstituted AML 3D BM niches derived from AML patients provided the optimal niche to support the primary AML in cultures. Specifically, the viability of AML cells co-cultured with the AML 3DMN and AML 3DON groups were the highest, while the percentages of apoptotic cells in them were the lowest. Cytokine array analysis revealed that the enhanced secretion of anti-apoptotic cytokines such as IL-8, GROα and OPG in the AML 3DMN and AML 3DON groups. In addition, the AML phenotype of CD33+ and CD34-were nicely preserved in these groups. The intrinsically high chemoresistance of AML was well retained by the AML niches (2DMN, 3DMN, and 3DON groups) as compared to the healthy niches and other control groups, using daunorubicin and doxorubicin as examples, suggesting the physiological resemblance of the AML niches and the necessity to reconstitute AML BM niches for valid drug screening. Moreover, the healthy BM niches (2DMN, 3DMN, and 3DON), while equally supporting the AML cell survival and phenotype maintenance in vitro, showed increased drug sensitivity to the two sample drugs used, indicating that reconstituting a healthy BM niche in leukemic patients may overcome the intrinsic chemoresistance problem in leukemia patients. This work contributes to the application of the AML 3DON in personalized drug screening and suggests the potential of using the healthy 3DON as a chemotherapeutic adjuvant for better AML chemotherapies with enhanced drug sensitivity.

FIG. 1C shows the workflow for high-throughput personalized drug screening. AML cells were isolated from patient blood sample and MSCs were isolated from patient or from other AML patient donors' blood sample. Robotic dispenser was utilized for the fabrication of 3DMN and 3DON in the 384-well plate such that the fabrication process can be carried out efficiently with high precision. AML cells were co-cultured with the 3DMN or 3DON in the presence of different types and dosage of drugs. The screening results were obtained by a microplate reader and analysed.

### REFERENCES

Aasebø, Elise et al. 2021. “Proteomic Characterization of Spontaneous Stress-Induced In Vitro Apoptosis of Human Acute Myeloid Leukemia Cells; Focus on Patient Heterogeneity and Endoplasmic Reticulum Stress.” Hemato 2021, Vol. 2, Pages 607-627 2 (3): 607-27. https://www.mdpi.com/2673-6357/2/3/39/htm (Mar. 17, 2022).
Abdul-Aziz, Amina M et al. “Microenvironment and Immunology MIF-Induced Stromal PKCb/IL8 Is Essential in Human Acute Myeloid Leukemia.”http://cancerres.aacrjournals.org/(May 19, 2022).
Antonelli, Antonella et al. 2016. “Establishing Human Leukemia Xenograft Mouse Models by Implanting Human Bone Marrow-like Scaffold-Based Niches.” Blood 128 (25): 2949-59.
Battula, Venkata Lokesh et al. 2017. “AML-Induced Osteogenic Differentiation in Mesenchymal Stromal Cells Supports Leukemia Growth.” JCI Insight 2 (13). /pmc/articles/PMC5499365/(Mar. 17, 2022).
Bertoli, Sarah et al. 2017. “Improved Outcome for AML Patients over the Years 2000-2014.” Blood Cancer Journal 2017 7:12 7 (12): 1-9. https://www.nature.com/articles/s41408-017-0011-1 (Mar. 15, 2022).
Blanco, Teresa Mortera, Athanasios Mantalaris, Alexander Bismarck, and Nicki Panoskaltsis. 2010. “The Development of a Three-Dimensional Scaffold for Ex Vivo Biomimicry of Human Acute Myeloid Leukaemia.” Biomaterials 31 (8): 2243-51.
Bray, Laura J. et al. 2017. “A Three-Dimensional Ex Vivo Tri-Culture Model Mimics Cell-Cell Interactions between Acute Myeloid Leukemia and the Vascular Niche.” Haematologica 102 (7): 1215-26. www.haematologica.org/content/102/7/1215 (Jul. 2, 2020).
Brenner, Annette K. et al. 2019. “The Capacity of Long-Term in Vitro Proliferation of Acute Myeloid Leukemia Cells Supported Only by Exogenous Cytokines Is Associated with a Patient Subset with Adverse Outcome.” Cancers 11 (1). https://pubmed.ncbi.nlm.nih.gov/30634713/(Mar. 18, 2022).
Brunetti, Lorenzo, Michael C. Gundry, and Margaret A. Goodell. 2019. “New Insights into the Biology of Acute Myeloid Leukemia with Mutated NPM1.” International Journal of Hematology 2019 110:2 110 (2): 150-60. https://link.springer.com/article/10.1007/s12185-018-02578-7 (Mar. 15, 2022).
Casasnovas, R. O. et al. 2003. “Immunological Classification of Acute Myeloblastic Leukemias: Relevance to Patient Outcome.” Leukemia 2003 17:3 17 (3): 515-27. https://www.nature.com/articles/2402821 (Jun. 14, 2022).
Chan, Barbara Pui et al. 2010. “Mesenchymal Stem Cell-Encapsulated Collagen Microspheres for Bone Tissue Engineering.” Tissue engineering. Part C, Methods 16 (2): 225-35. https://pubmed.ncbi.nlm.nih.gov/20367213/(Mar. 30, 2022).
Cucchi, D. G. J., R. W. J. Groen, J. J. W. M. Janssen, and J. Cloos. 2020. “Ex Vivo Cultures and Drug Testing of Primary Acute Myeloid Leukemia Samples: Current Techniques and Implications for Experimental Design and Outcome.” Drug Resistance Updates 53: 100730.
Duy, Cihangir et al. 2021. “Chemotherapy Induces Senescence-like Resilient Cells Capable of Initiating Aml Recurrence.” Cancer Discovery 11 (6): 1542-61. https://aacrjournals.org/cancerdiscovery/article/11/6/1542/666577/Chemotherapy-Induces-Senescence-Like-Resilient (Mar. 15, 2022).
van Gosliga, Djoke et al. 2007. “Establishing Long-Term Cultures with Self-Renewing Acute Myeloid Leukemia Stem/Progenitor Cells.” Experimental Hematology 35 (10): 1538-49.
Hogge, D. E. et al. 1996. “Enhanced Detection, Maintenance, and Differentiation of Primitive Human Hematopoietic Cells in Cultures Containing Murine Fibroblasts Engineered to Produce Human Steel Factor, Interleukin-3, and Granulocyte Colony-Stimulating Factor.” Blood 88 (10): 3765-73.
HoushmandMohammad et al. 2017. “Mimicking the Acute Myeloid Leukemia Niche for Molecular Study and Drug Screening.” https://home.liebertpub.com/tec 23 (2): 72-85. https://www.liebertpub.com/doi/abs/10.1089/ten.tec.2016.0404 (Sep. 7, 2021).
Kern, Wolfgang et al. 2010. “The Role of Multiparameter Flow Cytometry for Disease Monitoring in AML.” Best Practice & Research Clinical Haematology 23 (3): 379-90.
Khalidi, Hasan S. et al. 1998. “The Immunophenotype of Adult Acute Myeloid Leukemia: High Frequency of Lymphoid Antigen Expression and Comparison of Immunophenotype, French-American-British Classification, and Karyotypic Abnormalities.” American Journal of Clinical Pathology 109 (2): 211-20. https://academic.oup.com/ajcp/article/109/2/211/1757710 (Jun. 14, 2022).
Ladikou, E. E., H. Sivaloganathan, A. Pepper, and T. Chevassut. 2020. “Acute Myeloid Leukaemia in Its Niche: The Bone Marrow Microenvironment in Acute Myeloid Leukaemia.” Current Oncology Reports 22 (3): 1-9. https://link.springer.com/article/10.1007/s11912-020-0885-0 (Mar. 15, 2022).
Lam, Stephen S. Y. et al. 2016. “Homoharringtonine (Omacetaxine Mepesuccinate) as an Adjunct for FLT3-ITD Acute Myeloid Leukemia.” Science Translational Medicine 8 (359). https://www-science-org.eproxy.lib.hku.hk/doi/abs/10.1126/scitranslmed.aaf3735 (Mar. 17, 2022).
Marofi, Faroogh et al. 2021. “Novel CAR T Therapy Is a Ray of Hope in the Treatment of Seriously Ill AML Patients.” Stem Cell Research & Therapy 2021 12:1 12 (1): 1-23. https://link.springer.com/articles/10.1186/s13287-021-02420-8 (Mar. 15, 2022).
Plesa, A et al. 2008. 2 Math. Model. Nat. Phenom Diagnostics of the AML with Immunophenotypical Data.
Quek, Lynn et al. 2016. “Genetically Distinct Leukemic Stem Cells in Human CD34-Acute Myeloid Leukemia Are Arrested at a Hemopoietic Precursor-like Stage.” The Journal of experimental medicine 213 (8): 1513-35. https://pubmed.ncbi.nlm.nih.gov/27377587/(Mar. 17, 2022).
Ryningen, Anita et al. 2006. “Stress-Induced in Vitro Apoptosis of Native Human Acute Myelogenous Leukemia (AML) Cells Shows a Wide Variation between Patients and Is Associated with Low BCL-2:Bax Ratio and Low Levels of Heat Shock Protein 70 and 90.” Leukemia Research 30 (12): 1531-40.
Schuringa, Jan Jacob, and Hein Schepers. 2009. “Ex Vivo Assays to Study Self-Renewal and Long-Term Expansion of Genetically Modified Primary Human Acute Myeloid Leukemia Stem Cells.” Methods in Molecular Biology 538:287-300. https://link-springer-com.eproxy.lib.hku.hk/protocol/10.1007/978-1-59745-418-6_14 (Mar. 18, 2022).
Shafat, Manar S., Bruno Gnaneswaran, Kristian M. Bowles, and Stuart A. Rushworth. 2017. “The Bone Marrow Microenvironment—Home of the Leukemic Blasts.” Blood Reviews 31 (5): 277-86.
Stanchina, Michele et al. 2020. “Advances in Acute Myeloid Leukemia: Recently Approved Therapies and Drugs in Development.” Cancers 12 (11): 1-32. https://pubmed.ncbi.nlm.nih.gov/33139625/(Mar. 15, 2022).
Vetrie, David, G. Vignir Helgason, and Mhairi Copland. 2020. “The Leukaemia Stem Cell: Similarities, Differences and Clinical Prospects in CML and AML.” Nature Reviews Cancer 2020 20:3 20 (3): 158-73. https://www-nature-com.eproxy.lib.hku.hk/articles/s41568-019-0230-9 (Mar. 15, 2022).
Villatoro, Alicia, Joanna Konieczny, Vincent Cuminetti, and Lorena Arranz. 2020. “Leukemia Stem Cell Release From the Stem Cell Niche to Treat Acute Myeloid Leukemia.” Frontiers in Cell and Developmental Biology 8:607.
Wang, Andi, and Hua Zhong. 2018. “Roles of the Bone Marrow Niche in Hematopoiesis, Leukemogenesis, and Chemotherapy Resistance in Acute Myeloid Leukemia.” Hematology (Amsterdam, Netherlands) 23 (10): 729-39. https://pubmed.ncbi.nlm.nih.gov/29902132/(Mar. 17, 2022).
Yeung, Cecilia C. S., and Jerald Radich. 2017. “Predicting Chemotherapy Resistance in AML.” Current Hematologic Malignancy Reports 12 (6): 530-36. https://link.springer.com/article/10.1007/s11899-017-0378-x (Mar. 23, 2021).
Zeijlemaker, W., J. W. Gratama, and G. J. Schuurhuis. 2014. “Tumor Heterogeneity Makes AML a ‘Moving Target’ for Detection of Residual Disease.” Cytometry Part B: Clinical Cytometry 86 (1): 3-14. https://onlinelibrary.wiley.com/doi/full/10.1002/cyto.b.21134 (Jun. 5, 2022).
[1] L. Brunetti, M. C. Gundry, M. A. Goodell, New insights into the biology of acute myeloid leukemia with mutated NPM1, Int. J. Hematol. 2019 1102 110 (2019) 150-160. https://doi.org/10.1007/S12185-018-02578-7.
[2] C. Duy, M. Li, M. Teater, C. Meydan, F. E. Garrett-Bakelman, T. C. Lee, C. R. Chin, C. Durmaz, K. C. Kawabata, E. Dhimolea, C. S. Mitsiades, H. Doehner, R. J. D'andrea, M. W. Becker, E. M. Paietta, C. E. Mason, M. Carroll, A. M. Melnick, Chemotherapy induces senescence-like resilient cells capable of initiating aml recurrence, Cancer Discov. 11 (2021) 1542-1561. https://doi.org/10.1158/2159-8290.CD-20-1375/333525/AM/CHEMOTHERAPY-INDUCES-SENESCENCE-LIKE-RESILIENT.
[3] F. Marofi, H. S. Rahman, Z. M. J. Al-Obaidi, A. T. Jalil, W. K. Abdelbasset, W. Suksatan, A. E. Dorofeev, N. Shomali, M. S. Chartrand, Y. Pathak, A. Hassanzadeh, B. Baradaran, M. Ahmadi, H. Saeedi, S. Tahmasebi, M. Jarahian, Novel CAR T therapy is a ray of hope in the treatment of seriously ill AML patients, Stem Cell Res. Ther. 2021 121 12 (2021) 1-23. https://doi.org/10.1186/S13287-021-02420-8.
[4] D. Vetrie, G. V. Helgason, M. Copland, The leukaemia stem cell: similarities, differences and clinical prospects in CML and AML, Nat. Rev. Cancer 2020 203 20 (2020) 158-173. https://doi.org/10.1038/S41568-019-0230-9.
[5] M. Stanchina, D. Soong, B. Zheng-Lin, J. M. Watts, J. Taylor, Advances in Acute Myeloid Leukemia: Recently Approved Therapies and Drugs in Development, Cancers (Basel). 12 (2020) 1-32. https://doi.org/10.3390/CANCERS12113225.
[6] HoushmandMohammad, SoleimaniMasoud, AtashiAmir, SaglioGiuseppe, AbdollahiMohammad, N. ZarifMahin, Mimicking the Acute Myeloid Leukemia Niche for Molecular Study and Drug Screening, Https://Home.Liebertpub.Com/Tec 23 (2017) 72-85. https://doi.org/10.1089/TEN.TEC.2016.0404.
[7] S. Bertoli, S. Tavitian, A. Huynh, C. Borel, S. Guenounou, I. Luquet, E. Delabesse, A. Sarry, G. Laurent, M. Attal, F. Huguet, E. Bérard, C. Récher, Improved outcome for AML patients over the years 2000-2014, Blood Cancer J. 2017 712 7 (2017) 1-9. https://doi.org/10.1038/s41408-017-0011-1.
[8] C. C. S. Yeung, J. Radich, Predicting Chemotherapy Resistance in AML, Curr. Hematol. Malig. Rep. 12 (2017) 530-536. https://doi.org/10.1007/s11899-017-0378-x.
[9] A. Villatoro, J. Konieczny, V. Cuminetti, L. Arranz, Leukemia Stem Cell Release From the Stem Cell Niche to Treat Acute Myeloid Leukemia, Front. Cell Dev. Biol. 8 (2020) 607. https://doi.org/10.3389/FCELL.2020.00607/BIBTEX.
[10] M. S. Shafat, B. Gnaneswaran, K. M. Bowles, S. A. Rushworth, The bone marrow microenvironment—Home of the leukemic blasts, Blood Rev. 31 (2017) 277-286. https://doi.org/10.1016/J.BLRE.2017.03.004.
[11] A. Wang, H. Zhong, Roles of the bone marrow niche in hematopoiesis, leukemogenesis, and chemotherapy resistance in acute myeloid leukemia, Hematology 23 (2018) 729-739. https://doi.org/10.1080/10245332.2018.1486064.
[12] E. E. Ladikou, H. Sivaloganathan, A. Pepper, T. Chevassut, Acute Myeloid Leukaemia in Its Niche: the Bone Marrow Microenvironment in Acute Myeloid Leukaemia, Curr. Oncol. Rep. 22 (2020) 1-9. https://doi.org/10.1007/S11912-020-0885-0/FIGURES/1.
[13] L. Quek, G. W. Otto, C. Garnett, L. Lhermitte, D. Karamitros, B. Stoilova, I. J. Lau, J. Doondeea, B. Doondeca, A. Kennedy, M. Metzner, N. Goardon, A. Ivey, C. Allen, R. Gale, B. Davies, A. Sternberg, S. Killick, H. Hunter, P. Cahalin, A. Price, A. Carr, M. Griffiths, P. Virgo, S. Mackinnon, D. Grimwade, S. Freeman, N. Russell, C. Craddock, A. Mead, A. Peniket, C. Porcher, P. Vyas, Genetically distinct leukemic stem cells in human CD34-acute myeloid leukemia are arrested at a hemopoietic precursor-like stage, J. Exp. Med. 213 (2016) 1513-1535. https://doi.org/10.1084/JEM.20151775.
[14] V. L. Battula, P. M. Le, J. C. Sun, K. Nguyen, B. Yuan, X. Zhou, S. Sonnylal, T. McQueen, V. Ruvolo, K. A. Michel, X. Ling, R. Jacamo, E. Shpall, Z. Wang, A. Rao, G. Al-Atrash, M. Konopleva, R. E. Davis, M. A. Harrington, C. W. Cahill, C. Bueso-Ramos, M. Andreeff, AML-induced osteogenic differentiation in mesenchymal stromal cells supports leukemia growth, JCI Insight 2 (2017). https://doi.org/10.1172/JCI.INSIGHT.90036.
[15] A. M. Abdul-Aziz, M. S. Shafat, T. K. Mehta, F. Di Palma, M. J. Lawes, S. A. Rushworth, K. M. Bowles, Microenvironment and Immunology MIF-Induced Stromal PKCb/IL8 Is Essential in Human Acute Myeloid Leukemia, (n.d.). https://doi.org/10.1158/0008-5472.CAN-16-1095.
[16] E. Aasebø, A. K. Brenner, M. Hernandez-Valladares, E. Birkeland, H. Reikvam, F. Selheim, F. S. Berven, Ø. Bruserud, Proteomic Characterization of Spontaneous Stress-Induced In Vitro Apoptosis of Human Acute Myeloid Leukemia Cells; Focus on Patient Heterogeneity and Endoplasmic Reticulum Stress, Hemato 2021, Vol. 2, Pages 607-627 2 (2021) 607-627. https://doi.org/10.3390/HEMATO2030039.
[17] A. K. Brenner, E. Aasebø, M. Hernandez-Valladares, F. Selheim, F. Berven, I. S. Grønningsæter, S. Bartaula-Brevik, Ø. Bruserud, The Capacity of Long-Term in Vitro Proliferation of Acute Myeloid Leukemia Cells Supported Only by Exogenous Cytokines Is Associated with a Patient Subset with Adverse Outcome, Cancers (Basel). 11 (2019). https://doi.org/10.3390/CANCERS11010073.
[18] D. G. J. Cucchi, R. W. J. Groen, J. J. W. M. Janssen, J. Cloos, Ex vivo cultures and drug testing of primary acute myeloid leukemia samples: Current techniques and implications for experimental design and outcome, Drug Resist. Updat. 53 (2020) 100730. https://doi.org/10.1016/J.DRUP.2020.100730.
[19] A. Ryningen, E. Ersvær, A. M. Øyan, K. H. Kalland, O. K. Vintermyr, B. T. Gjertsen, Ø. Bruserud, Stress-induced in vitro apoptosis of native human acute myelogenous leukemia (AML) cells shows a wide variation between patients and is associated with low BCL-2:Bax ratio and low levels of heat shock protein 70 and 90, Leuk. Res. 30 (2006) 1531-1540. https://doi.org/10.1016/J.LEUKRES.2006.02.014.
[20] S. S. Y. Lam, E. S. K. Ho, B. L. He, W. W. Wong, C. Y. Cher, N. K. L. Ng, C. H. Man, H. Gill, A. M. S. Cheung, H. W. Ip, C. C. So, J. Tamburini, C. W. E. So, D. N. Ho, C. H. Au, T. L. Chan, E. S. K. Ma, R. Liang, Y. L. Kwong, A. Y. H. Leung, Homoharringtonine (omacetaxine mepesuccinate) as an adjunct for FLT3-ITD acute myeloid leukemia, Sci. Transl. Med. 8 (2016). https://doi.org/10.1126/SCITRANSLMED.AAF3735/SUPPL_FILE/8-359RA129_TABLES_S3_S4_AND_S8.ZIP.
[21] J. J. Schuringa, H. Schepers, Ex Vivo Assays to Study Self-Renewal and Long-Term Expansion of Genetically Modified Primary Human Acute Myeloid Leukemia Stem Cells, Methods Mol. Biol. 538 (2009) 287-300. https://doi.org/10.1007/978-1-59745-418-6_14.
[22] D. van Gosliga, H. Schepers, A. Rizo, D. van der Kolk, E. Vellenga, J. J. Schuringa, Establishing long-term cultures with self-renewing acute myeloid leukemia stem/progenitor cells, Exp. Hematol. 35 (2007) 1538-1549. https://doi.org/10.1016/J.EXPHEM.2007.07.001.
[23] A. Antonelli, W. A. Noort, J. Jaques, B. De Boer, R. De Jong-Korlaar, A. Z. Brouwers-Vos, L. Lubbers-Aalders, J. F. Van Velzen, A. C. Bloem, H. Yuan, J. D. De Bruijn, G. J. Ossenkoppele, A. C. M. Martens, E. Vellenga, R. W. J. Groen, J. J. Schuringa, Establishing human leukemia xenograft mouse models by implanting human bone marrow-like scaffold-based niches, Blood 128 (2016) 2949-2959. https://doi.org/10.1182/BLOOD-2016-05-719021.
[24] T. M. Blanco, A. Mantalaris, A. Bismarck, N. Panoskaltsis, The development of a three-dimensional scaffold for ex vivo biomimicry of human acute myeloid leukaemia, Biomaterials 31 (2010) 2243-2251. https://doi.org/10.1016/J.BIOMATERIALS.2009.11.094.
[25] L. J. Bray, M. Binner, Y. Körner, M. Von Bonin, M. Bornhäuser, C. Werner, A three-dimensional ex vivo tri-culture model mimics cell-cell interactions between acute myeloid leukemia and the vascular niche, Haematologica 102 (2017) 1215-1226. https://doi.org/10.3324/haematol.2016.157883.
[26] D. E. Hogge, P. M. Lansdorp, D. Reid, B. Gerhard, C. J. Eaves, Enhanced Detection, Maintenance, and Differentiation of Primitive Human Hematopoietic Cells in Cultures Containing Murine Fibroblasts Engineered to Produce Human Steel Factor, Interleukin-3, and Granulocyte Colony-Stimulating Factor, Blood 88 (1996) 3765-3773. https://doi.org/10.1182/BLOOD.V88.10.3765.BLOODJOURNAL88103765.
[27] P. Chandran, Y. Le, Y. Li, M. Sabloff, J. Mehic, M. Rosu-Myles, D. S. Allan, Mesenchymal stromal cells from patients with acute myeloid leukemia have altered capacity to expand differentiated hematopoietic progenitors, Leuk. Res. 39 (2015) 486-493. https://doi.org/10.1016/J.LEUKRES.2015.01.013.
[28] Z. G. Zhao, Y. Liang, K. Li, W. M. Li, Q. B. Li, Z. C. Chen, P. Zou, Phenotypic and Functional Comparison of Mesenchymal Stem Cells Derived from the Bone Marrow of Normal Adults and Patients with Hematologic Malignant Diseases, Https://Home.Liebertpub.Com/Scd 16 (2007) 637-648. https://doi.org/10.1089/SCD.2007.0008.
[29] B. P. Chan, T. Y. Hui, C. W. Yeung, J. Li, I. Mo, G. C. F. Chan, Self-assembled collagen-human mesenchymal stem cell microspheres for regenerative medicine, Biomaterials 28 (2007) 4652-4666. https://doi.org/10.1016/j.biomaterials.2007.07.041.
[30] T. Y. Hui, K. M. C. Cheung, W. L. Cheung, D. Chan, B. P. Chan, In vitro chondrogenic differentiation of human mesenchymal stem cells in collagen microspheres: Influence of cell seeding density and collagen concentration, Biomaterials 29 (2008) 3201-3212. https://doi.org/10.1016/J.BIOMATERIALS.2008.04.001.
[31] B. P. Chan, T. Y. Hui, M. Y. Wong, K. H. K. Yip, G. C. F. Chan, Mesenchymal stem cell-encapsulated collagen microspheres for bone tissue engineering, Tissue Eng. Part C. Methods 16 (2010) 225-235. https://doi.org/10.1089/TEN.TEC.2008.0709.
[32] M. Yuan, C. W. Yeung, Y. Y. Li, H. Diao, K. M. C. Cheung, D. Chan, K. Cheah, P. B. Chan, Effects of nucleus pulposus cell-derived acellular matrix on the differentiation of mesenchymal stem cells, Biomaterials 34 (2013) 3948-3961. https://doi.org/10.1016/J.BIOMATERIALS.2013.02.004.
[33] P. Yeung, H. S. Sin, S. Chan, G. C. F. Chan, B. P. Chan, Microencapsulation of Neuroblastoma Cells and Mesenchymal Stromal Cells in Collagen Microspheres: A 3D Model for Cancer Cell Niche Study, PLoS One 10 (2015) e0144139. https://doi.org/10.1371/journal.pone.0144139.
[34] G. R. Fajardo-Orduña, E. Ledesma-Martínez, I. Aguiñiga-Sánchez, M. de L. Mora-García, B. Weiss-Steider, E. Santiago-Osorio, Inhibitors of chemoresistance pathways in combination with ara-c to overcome multidrug resistance in aml. A mini review, Int. J. Mol. Sci. 22 (2021). https://doi.org/10.3390/ijms22094955.
[35] S. Geyh, M. Rodríguez-Paredes, P. Jäger, C. Khandanpour, R. P. Cadeddu, J. Gutekunst, C. M. Wilk, R. Fenk, C. Zilkens, D. Hermsen, U. Germing, G. Kobbe, F. Lyko, R. Haas, T. Schroeder, Functional inhibition of mesenchymal stromal cells in acute myeloid leukemia, Leuk. 2016 303 30 (2015) 683-691. https://doi.org/10.1038/leu.2015.325.
[36] H. C. Blair, Q. C. Larrouture, Y. Li, H. Lin, D. Beer-Stoltz, L. Liu, R. S. Tuan, L. J. Robinson, P. H. Schlesinger, D. J. Nelson, Osteoblast differentiation and bone matrix formation in vivo and in vitro, Tissue Eng.—Part B Rev. 23 (2017) 268-280. https://doi.org/10.1089/ten.teb.2016.0454.
[37] S. Geyh, M. Rodríguez-Paredes, P. Jäger, C. Khandanpour, R. P. Cadeddu, J. Gutekunst, C. M. Wilk, R. Fenk, C. Zilkens, D. Hermsen, U. Germing, G. Kobbe, F. Lyko, R. Haas, T. Schroeder, Functional inhibition of mesenchymal stromal cells in acute myeloid leukemia, Leuk. 2016 303 30 (2015) 683-691. https://doi.org/10.1038/leu.2015.325.
[38] A. M. Gruszka, D. Valli, C. Restelli, M. Alcalay, Adhesion Deregulation in Acute Myeloid Leukaemia, Cells 8 (2019) 66. https://doi.org/10.3390/CELLS8010066.
[39] S. Ito, A. J. Barrett, A. Dutra, E. Pak, S. Miner, K. Keyvanfar, N. F. Hensel, K. Rezvani, P. Muranski, P. Liu, J. J. Melenhorst, A. Larochelle, Long term maintenance of myeloid leukemic stem cells cultured with unrelated human mesenchymal stromal cells, Stem Cell Res. 14 (2015) 95-104. https://doi.org/10.1016/J.SCR.2014.11.007.
[40] D. Naci, S. Berrazouane, F. Barabé, F. Aoudjit, Cell adhesion to collagen promotes leukemia resistance to doxorubicin by reducing DNA damage through the inhibition of Rac1 activation, Sci. Reports 2019 91 9 (2019) 1-13. https://doi.org/10.1038/s41598-019-55934-w.
[41] X. Lin, S. Patil, Y. G. Gao, A. Qian, The Bone Extracellular Matrix in Bone Formation and Regeneration, Front. Pharmacol. 11 (2020) 757. https://doi.org/10.3389/FPHAR.2020.00757/BIBTEX.
[42] E. Aasebø, E. Birkeland, F. Selheim, F. Berven, A. K. Brenner, Ø. Bruserud, The Extracellular Bone Marrow Microenvironment—A Proteomic Comparison of Constitutive Protein Release by In Vitro Cultured Osteoblasts and Mesenchymal Stem Cells, Cancers (Basel). 13 (2021) 1-24. https://doi.org/10.3390/CANCERS13010062.
[43] H. Hanna, L. Mir, F. André, L. M. Mir, F. M. Andre, In vitro osteoblastic differentiation of mesenchymal stem cells generates cell layers with distinct properties, Stem Cell Res. Ther. 9 (2018) 10. https://doi.org/10.1186/s13287-018-0942-xï.
[44] W. Y. Lai, Y. Y. Li, S. K. Mak, F. C. Ho, S. T. Chow, W. H. Chooi, C. H. Chow, A. Y. Leung, B. P. Chan, Reconstitution of bone-like matrix in osteogenically differentiated mesenchymal stem cell-collagen constructs: A three-dimensional in vitro model to study hematopoietic stem cell niche: Https://Doi.org/10.1177/2041731413508668 4 (2013) 1-14. https://doi.org/10.1177/2041731413508668.
[45] T. Chen, G. Zhang, L. Kong, S. Xu, Y. Wang, M. Dong, Leukemia-derived exosomes induced IL-8 production in bone marrow stromal cells to protect the leukemia cells against chemotherapy, Life Sci. 221 (2019) 187-195. https://doi.org/10.1016/J.LFS.2019.02.003.
[46] R. J. Bodnar, Chemokine Regulation of Angiogenesis During Wound Healing, Adv. Wound Care 4 (2015) 641. https://doi.org/10.1089/WOUND.2014.0594.
[47] E. C. de Alvarenga, W. N. Silva, R. Vasconcellos, E. J. Paredes-Gamero, A. Mintz, A. Birbrair, Promyelocytic leukemia protein in mesenchymal stem cells is essential for leukemia progression, Ann. Hematol. 97 (2018) 1749-1755. https://doi.org/10.1007/S00277-018-3463-X/FIGURES/1.
[48] H. Cao, V. Tadros, B. Hiramoto, K. Leeper, C. Hino, J. Xiao, B. Pham, D. H. Kim, M. E. Reeves, C. S. Chen, J. F. Zhong, K. K. Zhang, L. Xie, S. Wasnik, D. J. Baylink, Y. Xu, Targeting TKI-Activated NFKB2-MIF/CXCLs-CXCR2 Signaling Pathways in FLT3 Mutated Acute Myeloid Leukemia Reduced Blast Viability, Biomedicines 10 (2022) 1038. https://doi.org/10.3390/BIOMEDICINES10051038/S1.
[49] C. Schinke, O. Giricz, W. Li, A. Shastri, S. Gordon, L. Barreyro, T. Bhagat, S. Bhattacharyya, N. Ramachandra, M. Bartenstein, A. Pellagatti, J. Boultwood, A. Wickrema, Y. Yu, B. Will, S. Wei, U. Steidl, A. Verma, IL8-CXCR2 pathway inhibition as a therapeutic strategy against MDS and AML stem cells, Blood 125 (2015) 3144-3152. https://doi.org/10.1182/BLOOD-2015-01-621631.
[50] C. Schinke, O. Giricz, S. A. K. Gordon, L. Barreyro, T. D. Bhagat, A. Pellagatti, J. Boultwood, Y. Yu, U. Steidl, A. Verma, Inhibition Of CXCR2 As a Therapeutic Strategy In AML and MDS, Blood 122 (2013) 484. https://doi.org/10.1182/BLOOD.V122.21.484.484.
[51] P. Secchiero, G. Zauli, Tumor necrosis factor-related apoptosis-inducing ligand and the regulation of hematopoiesis, Curr. Opin. Hematol. 15 (2008) 42-48. https://doi.org/10.1097/MOH.0B013E3282F15FA6.
[52] R. M. Locklin, P. I. Croucher, R. G. G. Russell, C. M. Edwards, Agonists of TRAIL death receptors induce myeloma cell apoptosis that is not prevented by cells of the bone marrow microenvironment, Leukemia 21 (2007) 805-812. https://doi.org/10.1038/SJ.LEU.2404518.
[53] B. Yuan, S. Ly, K. Nguyen, V. Tran, K. Maldonado, C. Kinglsey, J. K. Burks, X. Zhou, B. deCrombrugghe, M. Andreeff, V. L. Battula, Acute Myeloid Leukemia Expands Osteoprogenitor Rich Niche in the Bone Marrow but Resorbs Mature Bone Causing Osteopenia/Osteoporosis in Animal Models, Blood 132 (2018) 86-86. https://doi.org/10.1182/blood-2018-99-117258.
[54] K. A. Goncalves, G.-F. Hu, Mechanism and Function of Angiogenin in Hematopoietic Malignancy, Zhongguo Sheng Wu Hua Xue Yu Fen Zi Sheng Wu Xue Bao=Chinese J. Biochem. Mol. Biol. 31 (2015) 1267. /pmc/articles/PMC4750500/(accessed Jun. 11, 2022).
[55] R. Durai, S. Y. Yang, K. M. Sales, A. M. Scifalian, G. Goldspink, M. C. Winslet, Insulin-like growth factor binding protein-4 gene therapy increases apoptosis by altering Bcl-2 and Bax proteins and decreases angiogenesis in colorectal cancer, Int. J. Oncol. 30 (2007) 883-888. https://doi.org/10.3892/IJO.30.4.883/HTML.
[56] H. Reikvam, K. J. Hatfield, A. M. Øyan, K. H. Kalland, A. O. Kittang, Ø. Bruserud, Primary human acute myelogenous leukemia cells release matrix metalloproteases and their inhibitors: Release profile and pharmacological modulation, Eur. J. Haematol. 84 (2010) 239-251. https://doi.org/10.1111/J.1600-0609.2009.01382.X.
[57] L. I. Lin, D. T. Lin, C. J. Chang, C. Y. Lee, J. L. Tang, H. F. Tien, Marrow matrix metalloproteinases (MMPS) and tissue inhibitors of MMP in acute leukaemia: Potential role of MMP-9 as a surrogate marker to monitor leukaemic status in patients with acute myelogenous leukaemia, Br. J. Haematol. 117 (2002) 835-841. https://doi.org/10.1046/j.1365-2141.2002.03510.x.
[58] C. Wang, Z. Chen, Z. Li, J. Cen, The essential roles of matrix metalloproteinase-2, membrane type 1 metalloproteinase and tissue inhibitor of metalloproteinase-2 in the invasive capacity of acute monocytic leukemia SHI-1 cells, Leuk. Res. 34 (2010) 1083-1090. https://doi.org/10.1016/J.LEUKRES.2010.01.016.
[59] M. Luciano, P. W. Krenn, J. Horejs-Hoeck, The cytokine network in acute myeloid leukemia, Front. Immunol. 13 (2022). https://doi.org/10.3389/FIMMU.2022.1000996.
[60] K. N. Kremer, A. Dudakovic, M. E. Mcgee-Lawrence, R. L. Philips, A. D. Hess, B. D. Smith, A. J. Van Wijnen, J. E. Karp, S. H. Kaufmann, J. J. Westendorf, K. E. Hedin, Osteoblasts Protect AML Cells From SDF-1-Induced Apoptosis, J. Cell. Biochem 115 (2014) 1128-1137. https://doi.org/10.1002/jcb.24755.
[61] B. Landry, H. Gül-Uludal, S. Plianwong, C. Kucharski, Z. Zak, M. B. Parmar, O. Kutsch, H. Jiang, J. Brandwein, H. Uludal, Targeting CXCR4/SDF-1 axis by lipopolymer complexes of siRNA in acute myeloid leukemia, J. Control. Release 224 (2016) 8-21. https://doi.org/10.1016/J.JCONREL.2015.12.052.
[62] P. Bianchi, M. H. Séguélas, A. Parini, C. Cambon, Activation of Pro-Apoptotic Cascade by Dopamine in Renal Epithelial Cells is Fully Dependent on Hydrogen Peroxide Generation by Monoamine Oxidases, J. Am. Soc. Nephrol. 14 (2003) 855-862. https://doi.org/10.1097/01.ASN.0000058909.00567.5C.
[63] S. Zhang, Y. Zhang, Y. Zhuang, J. Wang, J. Ye, S. Zhang, J. Wu, K. Yu, Y. Han, Matrine Induces Apoptosis in Human Acute Myeloid Leukemia Cells via the Mitochondrial Pathway and Akt Inactivation, PLoS One 7 (2012) e46853. https://doi.org/10.1371/JOURNAL.PONE.0046853.
[64] I. Tagoug, A. S. de Chalon, C. Dumontet, Inhibition of IGF-1 Signalling Enhances the Apoptotic Effect of AS602868, an IKK2 Inhibitor, in Multiple Myeloma Cell Lines, PLoS One 6 (2011) c22641. https://doi.org/10.1371/JOURNAL.PONE.0022641.
[65] S. Iqbal, S. Zhang, A. Driss, Z. R. Liu, H. R. C. Kim, Y. Wang, C. Ritenour, H. E. Zhau, O. Kucuk, L. W. K. Chung, D. Wu, PDGF upregulates Mc1-1 through activation of β-catenin and HIF-1α-dependent signaling in human prostate cancer cells, PLoS One 7 (2012). https://doi.org/10.1371/JOURNAL.PONE.0030764.
[66] S. P. Glaser, E. F. Lee, E. Trounson, P. Bouillet, A. Wei, W. D. Fairlie, D. J. Izon, J. Zuber, A. R. Rappaport, M. J. Herold, W. S. Alexander, S. W. Lowe, L. Robb, A. Strasser, Anti-apoptotic Mc1-1 is essential for the development and sustained growth of acute myeloid leukemia, Genes Dev. 26 (2012) 120. https://doi.org/10.1101/GAD.182980.111.
[67] G. Hoermann, S. Cerny-Reiterer, H. Herrmann, K. Blatt, M. Bilban, H. Gisslinger, B. Gisslinger, L. Müllauer, R. Kralovics, C. Mannhalter, P. Valent, M. Mayerhofer, Identification of oncostatin M as a JAK2 V617F-dependent amplifier of cytokine production and bone marrow remodeling in myeloproliferative neoplasms, FASEB J. •Res. Commun. (n.d.). https://doi.org/10.1096/fj.11-193078.
[68] P. S. Becker, Dependence of acute myeloid leukemia on adhesion within the bone marrow microenvironment, Scientific WorldJournal. 2012 (2012) 9-12. https://doi.org/10.1100/2012/856467.
[69] C. Gabay, Interleukin-6 and chronic inflammation, Arthritis Res. Ther. 8 (2006) 1-6. https://doi.org/10.1186/ar1917.
[70] E. Sano, A. Kazaana, H. Tadakuma, T. Takei, S. Yoshimura, Y. Hanashima, Y. Ozawa, A. Yoshino, Y. Suzuki, T. Ueda, Interleukin-6 sensitizes TNF-α and TRAIL/Apo2L dependent cell death through upregulation of death receptors in human cancer cells, Biochim. Biophys. Acta-Mol. Cell Res. 1868 (2021) 119037. https://doi.org/10.1016/J.BBAMCR.2021.119037.
[71] M. Pomaznoy, B. Ha, B. Peters, GOnet: A tool for interactive Gene Ontology analysis, BMC Bioinformatics 19 (2018) 1-8. https://doi.org/10.1186/s12859-018-2533-3.
[72] W. Kern, U. Bacher, C. Haferlach, S. Schnittger, T. Haferlach, The role of multiparameter flow cytometry for disease monitoring in AML, Best Pract. Res. Clin. Haematol. 23 (2010) 379-390. https://doi.org/10.1016/J.BEHA.2010.06.007.
[73] W. Zeijlemaker, J. W. Gratama, G. J. Schuurhuis, Tumor heterogeneity makes AML a “moving target” for detection of residual disease, Cytom. Part B Clin. Cytom. 86 (2014) 3-14. https://doi.org/10.1002/CYTO.B.21134.
[74] A. Plesa, G. Ciuperca, V. Louvet, L. Pujo-Menjouet, S. Génieys, C. Dumontet, X. Thomas, V. Volpert, Diagnostics of the AML with immunophenotypical data, 2008.
[75] H. S. Khalidi, L. J. Medeiros, K. L. Chang, R. K. Brynes, M. L. Slovak, D. A. Arber, The Immunophenotype of Adult Acute Myeloid Leukemia: High Frequency of Lymphoid Antigen Expression and Comparison of Immunophenotype, French-American-British Classification, and Karyotypic Abnormalities, Am. J. Clin. Pathol. 109 (1998) 211-220. https://doi.org/10.1093/AJCP/109.2.211.
[76] R. O. Casasnovas, F. K. Slimane, R. Garand, G. C. Faure, L. Campos, V. Deneys, M. Bernier, A. Falkenrodt, G. Lecalvez, M. Maynadié, M. C. Béné, Immunological classification of acute myeloblastic leukemias: relevance to patient outcome, Leuk. 2003 173 17 (2003) 515-527. https://doi.org/10.1038/sj.leu.2402821.
[77] L. C. Marti, N. S. Bacal, L. C. Bento, F. A. Rocha, Phenotypic Markers and Functional Regulators of Myelomonocytic Cells, Biol. Myelomonocytic Cells (2017) 3-20. https://doi.org/10.5772/67324.
[78] P. Maiso, E. Colado, E. M. Ocio, M. Garayoa, J. Martín, P. Atadja, A. Pandiella, J. F. San-Miguel, The synergy of panobinostat plus doxorubicin in acute myeloid leukemia suggests a role for HDAC inhibitors in the control of DNA repair, Leuk. 2009 2312 23 (2009) 2265-2274. https://doi.org/10.1038/leu.2009.182.
[79] J. Jin, X. Li, W. Guo, F. Li, J. Huang, X. Huang, J. Pan, S. Huang, W. Ye, Q. Ling, J. Jin, Novel SAHA-bendamustine hybrid NL-101 in combination with daunorubicin synergistically suppresses acute myeloid leukemia, Oncol. Rep. 44 (2020) 273-282. https://doi.org/10.3892/OR.2020.7591/HTML.
[80] S. Karathedath, B. M. Rajamani, S. M. M. Aalam, A. Abraham, S. Varatharajan, P. Krishnamurthy, V. Mathews, S. R. Velayudhan, P. Balasubramanian, Role of NF-E2 related factor 2 (Nrf2) on chemotherapy resistance in acute myeloid leukemia (AML) and the effect of pharmacological inhibition of Nrf2, PLoS One 12 (2017). https://doi.org/10.1371/JOURNAL.PONE.0177227.
[81] O. A. Guryanova, K. Shank, B. Spitzer, L. Luciani, R. P. Koche, F. E. Garrett-Bakelman, C. Ganzel, B. H. Durham, A. Mohanty, G. Hoermann, S. A. Rivera, A. G. Chramiec, E. Pronier, L. Bastian, M. D. Keller, D. Tovbin, E. Loizou, A. R. Weinstein, A. R. Gonzalez, Y. K. Lieu, J. M. Rowe, F. Pastore, A. S. McKenney, A. V. Krivtsov, W. R. Sperr, J. R. Cross, C. E. Mason, M. S. Tallman, M. E. Arcila, O. Abdel-Wahab, S. A. Armstrong, S. Kubicek, P. B. Staber, M. Gönen, E. M. Paietta, A. M. Melnick, S. D. Nimer, S. Mukherjee, R. L. Levine, DNMT3A mutations promote anthracycline resistance in acute myeloid leukemia via impaired nucleosome remodeling, Nat. Med. 22 (2016) 1488-1495. https://doi.org/10.1038/nm.4210.
[82] L. Qiu, G. Zhou, S. Cao, Targeted inhibition of ULK1 enhances daunorubicin sensitivity in acute myeloid leukemia, Life Sci. 243 (2020) 117234. https://doi.org/10.1016/j.lfs.2019.117234.
[83] M. Kobyakova, Y. Lomovskaya, A. Senotov, A. Lomovsky, V. Minaychev, I. Fadeeva, D. Shtatnova, K. Krasnov, A. Zvyagina, I. Odinokova, V. Akatov, R. Fadeev, The Increase in the Drug Resistance of Acute Myeloid Leukemia THP-1 Cells in High-Density Cell Culture Is Associated with Inflammatory-like Activation and Anti-Apoptotic Bcl-2 Proteins, Int. J. Mol. Sci. 23 (2022) 7881. https://doi.org/10.3390/IJMS23147881/S1.
[84] J. Varshosaz, F. Hassanzadch, H. Sadeghi Aliabadi, M. Nayebsadrian, M. Banitalebi, M. Rostami, Synthesis and characterization of folate-targeted dextran/retinoic acid micelles for doxorubicin delivery in acute leukemia, Biomed Res. Int. 2014 (2014). https://doi.org/10.1155/2014/525684.
[85] M. Sharma, C. Ross, S. Srivastava, Ally to adversary: Mesenchymal stem cells and their transformation in leukaemia, Cancer Cell Int. 19 (2019) 1-9. https://doi.org/10.1186/s12935-019-0855-5.
[86] H. Koyama, N. Kobayashi, M. Harada, M. Takcoka, Y. Kawai, K. Sano, M. Fujimori, J. Amano, T. Ohhashi, R. Kannagi, K. Kimata, S. Taniguchi, N. Itano, Significance of tumor-associated stroma in promotion of intratumoral lymphangiogenesis: Pivotal role of a hyaluronan-rich tumor microenvironment, Am. J. Pathol. 172 (2008) 179-193. https://doi.org/10.2353/ajpath.2008.070360.
[87] K. T. Hua, W. J. Lec, S. F. Yang, C. K. Chen, M. Hsiao, C. C. Ku, L. H. Wei, M. L. Kuo, M. H. Chien, Vascular endothelial growth factor-C modulates proliferation and chemoresistance in acute myeloid leukemic cells through an endothelin-1-dependent induction of cyclooxygenase-2, Biochim. Biophys. Acta-Mol. Cell Res. 1843 (2014) 387-397. https://doi.org/10.1016/J.BBAMCR.2013.10.015.
[88] R. M. Sterner, K. N. Kremer, A. Dudakovic, J. J. Westendorf, A. J. van Wijnen, K. E. Hedin, Tissue-Nonspecific Alkaline Phosphatase Is Required for MC3T3 Osteoblast—Mediated Protection of Acute Myeloid Leukemia Cells from Apoptosis, J. Immunol. 201 (2018) 1086-1096. https://doi.org/10.4049/JIMMUNOL.1800174.
[89] D. R. Chen, D. Y. Lu, H. Y. Lin, W. L. Yeh, Mesenchymal stem cell-induced doxorubicin resistance in triple negative breast cancer, Biomed Res. Int. 2014 (2014). https://doi.org/10.1155/2014/532161.
[90] R. Granata, L. Trovato, G. Garbarino, M. Taliano, R. Ponti, G. Sala, R. Ghidoni, E. Ghigo, Dual effects of IGFBP-3 on endothelial cell apoptosis and survival: Involvement of the sphingolipid signaling pathways, FASEB J. (2004). https://doi.org/10.1096/fj.04-1618fje.
[91] D. Y. Lee, H. K. Yi, P. H. Hwang, Y. Oh, Enhanced expression of insulin-like growth factor binding protein-3 sensitizes the growth inhibitory effect of anticancer drugs in gastric cancer cells, Biochem. Biophys. Res. Commun. 294 (2002) 480-486. https://doi.org/10.1016/S0006-291X (02) 00491-6.
[92] R. Granata, M. De Petrini, L. Trovato, R. Ponti, N. Pons, C. Ghè, A. Graziani, R. J. Ferry, G. Muccioli, E. Ghigo, Insulin-like growth factor binding protein-3 mediates serum starvation- and doxorubicin-induced apoptosis in H9c2 cardiac cells, J. Endocrinol. Invest. 26 (2003) 1231-1241. https://doi.org/10.1007/BF03349163.
[93] B. J. Pettus, C. E. Chalfant, Y. A. Hannun, Ceramide in apoptosis: An overview and current perspectives, Biochim. Biophys. Acta-Mol. Cell Biol. Lipids 1585 (2002) 114-125. https://doi.org/10.1016/S1388-1981 (02) 00331-1.
[94] J. C. Huang, S. K. Basu, X. Zhao, S. Chien, M. Fang, V. G. Oehler, F. R. Appelbaum, P. S. Becker, Mesenchymal stromal cells derived from acute myeloid leukemia bone marrow exhibit aberrant cytogenetics and cytokine elaboration, Blood Cancer J. 2015 54 5 (2015) e302-e302. https://doi.org/10.1038/bcj.2015.17.
[95] S. Geyh, M. Rodríguez-Paredes, P. Jäger, C. Khandanpour, R. P. Cadeddu, J. Gutekunst, C. M. Wilk, R. Fenk, C. Zilkens, D. Hermsen, U. Germing, G. Kobbe, F. Lyko, R. Haas, T. Schroeder, Functional inhibition of mesenchymal stromal cells in acute myeloid leukemia, Leuk. 2016 303 30 (2015) 683-691. https://doi.org/10.1038/leu.2015.325.
[96] P. T. Kamga, G. Bassi, A. Cassaro, M. Midolo, M. Di Trapani, A. Gatti, R. Carusone, F. Resci, O. Perbellini, M. Gottardi, M. Bonifacio, A. H. N. Kamdje, A. Ambrosetti, M. Krampera, Notch signalling drives bone marrow stromal cell-mediated chemoresistance in acute myeloid leukemia, Oncotarget 7 (2016) 21713. https://doi.org/10.18632/ONCOTARGET.7964.
[97] R. C. Schelker, S. Iberl, G. Müller, C. Hart, W. Herr, J. Grassinger, TGF-β1 and CXCL12 modulate proliferation and chemotherapy sensitivity of acute myeloid leukemia cells co-cultured with multipotent mesenchymal stromal cells, Https://Doi.org/10.1080/10245332.2017.1402455 23 (2017) 337-345. https://doi.org/10.1080/10245332.2017.1402455.
[98] J. Lu, Q. Dong, S. Zhang, Y. Feng, J. Yang, L. Zhao, Acute myeloid leukemia (AML)-derived mesenchymal stem cells induce chemoresistance and epithelial-mesenchymal transition-like program in AML through IL-6/JAK2/STAT3 signaling, Cancer Sci. (2023) 1-14. https://doi.org/10.1111/cas.15855.
[99] M. Almosailleakh, J. Schwaller, Murine models of acute Myeloid Leukaemia, Int. J. Mol. Sci. 20 (2019). https://doi.org/10.3390/ijms20020453.
[100] H. Skayneh, B. Jishi, R. Hleihel, M. Hamieh, N.D. 4, A Critical Review of Animal Models Used in Acute Myeloid Myeloid Leukemia Pathophysiology, Juornal Mdpi 1 (2020) 1-35.
[101] W. Y. Lai, Y. Y. Li, S. K. Mak, F. C. Ho, S. T. Chow, W. H. Chooi, C. H. Chow, A. Y. Leung, B. P. Chan, Reconstitution of bone-like matrix in osteogenically differentiated mesenchymal stem cell-collagen constructs: A three-dimensional in vitro model to study hematopoietic stem cell niche: Https://Doi.org/10.1177/2041731413508668 4 (2013) 1-14. https://doi.org/10.1177/2041731413508668.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Those skilled in the art will recognize or be able to ascertain using no more than routine experimentation, many equivalents to the specific embodiments of the method and compositions described herein. Such equivalents are intended to be encompassed by the following claims.

We claim:

1. A biomimetic 3D osteogenic niche (3DON) formed by encapsulating mesenchymal stromal cells (MSCs) in extracellular matrix (ECM) and differentiating the MSCs towards the osteogenic lineage.

2. The 3DON of claim 1, wherein the MSCs are bone marrow derived MSCs.

3. The 3DON of claim 1, wherein the MSCs are osteoblast cells, osteoclast cells, osteocytes, endothelial cells, adipocytes, immune cells, or combinations thereof.

4. The 3DON of claim 1, wherein the ECM is collagen, fibronectin, laminin, Matrigel, or combinations thereof.

5. The 3DON of claim 1, wherein the 3DON is formed by:
(a) mixing the ECM, the MSCs, sodium hydroxide, and culture medium in a specific order, ratio, pH, concentration, and volume, wherein the mixing is performed in in a culture plate coated with pluronic F127, wherein the temperature and incubation time are chosen to allow for gelation, and
(b) inducing osteogenic differentiation by use of osteogenic induction medium prepared with dexamethasone, β-glycerophosphate, ascorbic acid, and bone morphogenic protein 2 (BMP2) in a specific concentration.

6. A platform comprising leukemia cells co-cultured with 3DON of claim 1, wherein the platform supports the survival of the leukemia cells.

7. The platform of claim 6, wherein the leukemia cells are primary acute myeloid leukemia (AML).

8. The platform of claim 6, wherein co-culturing the 3DON and leukemia cells supports the survival, reduce the apoptosis, and/or maintain the phenotype of the leukemia cells.

9. The platform of claim 6, wherein co-culturing the 3DON and leukemia cells increases the chemo-sensitivity of leukemia cells.

10. The platform of claim 6, wherein the percentage of viable leukemia cells is increased and/or and the percentage of apoptotic leukemia cells is reduced in leukemia cells maintained in the platform as compared to leukemia cells not maintained in the platform.

11. The platform of claim 6, wherein the phenotype of primary cells (CD33+ and CD34−) is preserved to a greater extent in the cells maintained in the platform as compared to cells not maintained in the platform.

12. A method of drug screening comprising exposing the leukemia cells of the platform of claim 6 to a test compound and comparing the effect of the test compound on the leukemia cells to a control platform that was not exposed to the drug.

13. A method comprising treating a patient in need of chemotherapy with a chemotherapeutic and with the 3DON of claim 1.

14. The method of claim 13, wherein the 3DON is in a pharmaceutical formulation.
